(19) United States
(12) Patent Application Publication
(10) Pub. No.: US 2014/0370084 A1
(43) Pub. Date: Dec. 18, 2014

(54) ESTRADIOL FORMULATIONS AND THERAPIES

(71) Applicant: TherapeuticsMD, Inc., Boca Raton, FL (US)

(72) Inventors: Brian A. Bernick, Boca Raton, FL (US); Janice Louise Cacace, Miami, FL (US); Peter H.R. Persicaner, Boca Raton, FL (US); Neda Irani, Palm Beach Garden, FL (US); Julia M. Amadio, Boca Raton, FL (US)

(73) Assignee: TherapeuticsMD, Inc., Boca Raton, FL (US)

(21) Appl. No.: 14/106,655

(22) Filed: Dec. 13, 2013

Related U.S. Application Data
(63) Continuation of application No. PCT/US2013/046445, filed on Jun. 18, 2013.

(51) Int. Cl. A61K 31/565

(52) U.S. Cl. CPC A61K 31/565
USPC 424/456; 514/182; 424/451

(57) ABSTRACT

Estrogen and progesterone replacement therapies are provided herein. Among others, the following formulations are provided herein: solubilized estradiol without progesterone; micronized progesterone without estradiol; micronized progesterone with partially solubilized progesterone; solubilized estradiol with micronized progesterone; solubilized estradiol with micronized progesterone in combination with partially solubilized progesterone; and solubilized estradiol with solubilized progesterone.

### CROSS REFERENCE TO RELATED APPLICATIONS

The present application is a continuation of International Patent Application No. PCT/US13/46445, entitled “NATURAL COMBINATION HORMONE REPLACEMENT FORMULATIONS AND THERAPIES,” which was filed on Jun. 18, 2013; and the present application claims priority to the following U.S. patent applications: U.S. patent application Ser. No. 13/843,428, entitled “NATURAL COMBINATION HORMONE REPLACEMENT FORMULATIONS AND THERAPIES,” which was filed on Mar. 15, 2013; U.S. application Ser. No. 13/684,002, entitled “NATURAL COMBINATION HORMONE REPLACEMENT THERAPIES,” which was filed on Nov. 21, 2012; U.S. Provisional Application Ser. No. 61/661,302, entitled “ESTRADIOL FORMULATIONS,” which was filed on Jun. 18, 2012; U.S. Provisional Application Ser. No. 61/662,265, entitled “PROGESTERONE FORMULATIONS,” which was filed on Jun. 20, 2012; and U.S. Provisional Application Ser. No. 61/563,408, entitled “NATURAL COMBINATION HORMONE REPLACEMENT THERAPIES,” which was filed on Nov. 23, 2011; which applications are incorporated by reference herein in their entirety.

### BACKGROUND

1. Field

This disclosure relates to natural estrogen and progesterone replacement therapies, with formulations provided for each estradiol and progesterone alone and in combination for the treatment of pre-menopausal, peri-menopausal, menopausal and post-menopausal females in relation to the treatment of Estrogen- and Progesterone-deficient states, each as herein below defined.

2. Discussion of the Related Art

Hormone Replacement Therapy (HRT) is a medical treatment that involves the use of one or more of a group of medications designed to increase hormone levels in women who lack adequate hormone production. HRT can mitigate and prevent symptoms caused by diminished circulating estrogen and progesterone hormones regardless as to whether the subject is pre-menopausal, peri-menopausal, menopausal or post-menopausal. However, specific symptomatic states can exist during each stage of menopausal progression.

HRT is presently available in various forms. One therapy involves administration of low dosages of one or more estrogens. Another involves administration of progesterone or a chemical analogue, called a progestin. Progesterone administration acts, among treating other disease states, to mitigate certain undesirable side effects from estrogen administration including, for example, endometrial hyperplasia (thickening) and reducing the incidence of endometrial cancer.

Timing for dosage administration is often varied cyclically, with estrogens taken daily and progesterone taken for approximately two weeks of every month, a method often referred to as “Cyclic-Sequential” or “Sequentially-Combined HRT.” This method is intended to mimic the natural menstrual cycle and typically causes menstruation similar to a period after the progesterone is stopped. This regimen is most typically used in peri-menopausal or newly menopausal women as the alternative continuous method often results in irregular bleeding in such women. An alternate method, a constant dosage with both estrogen and progesterone taken daily, is called “Continuous-Combined HRT.” This method usually results in no menstruation and is used most often after a woman has been menopausal for some time.

Estrogen, in its various forms, and progesterone, in its various forms, are used in HRT via a variety of administered dosage forms including, for example, via tablets, capsules and patches.

“Bio-identical” hormones, which are identical in chemical structure to the hormones naturally produced by human bodies can be used and are often referred to as Natural Hormone Replacement Therapy or NHRT.

These natural or bio-identical hormones are formulated from various ingredients to match the chemical structure and effect of estradiol, estrone, or estriol (the 3 primary estrogens) as well as progesterone that occurs naturally in the human body (endogenous).

Currently, bio-identical estradiol is available in both branded and generic FDA approved versions. FDA-approved bio-identical progesterone for HRT is available as the branded stand-alone drug commercially identified as PROMETRIUM (progesterone, USP) (Abbott Laboratories, Abbott Park, Ill.), with a generic authorized by the innovator, and generic products provided by Teva (Israel) and Sofgen Americas, Inc. (New York). PROMETRIUM was approved for sale in the United States on May 14, 1998 under NDA # N019781. According to the prescribing information approved for this product (Rev June 2009) (“PROMETRIUM prescribing information”), PROMETRIUM comprises synthetic progesterone that is chemically identical to progesterone of human ovarian origin. Capsules comprise 100 mg or 200 mg of micronized progesterone. The inactive ingredients include peanut oil, gelatin, glycerin, lecithin, titanium dioxide, and yellow and red dyes.

Other products such as PREMPRO (conjugated estrogens/medroxyprogesterone acetate tablets) and PREMPHASE (conjugated estrogens plus medroxyprogesterone acetate tablets) (Wyeth Laboratories, a division of Pfizer, Inc., New York) provide both continuous-combined and cyclic-sequential products containing PREMARIN (estrogen derived from mare's urine) and synthetic medroxyprogesterone acetate. Other products are available. However, no FDA approved product exists on the market today with combination bio-identical estradiol and bio-identical progesterone.

### SUMMARY

According to various embodiments of the disclosure, natural hormone replacement therapies are provided comprising cyclic-sequential and continuous-combined delivery via pharmaceutical formulations of solubilized estradiol and micronized and/or partially or completely solubilized progesterone. Estradiol and micronized and/or partially or completely solubilized progesterone delivered together daily can be combined in either a single unit dose or in separate unit doses, typically in a soft capsule. In some embodiments, pharmaceutical formulations are disclosed comprising solubilized estradiol. In some embodiments, the estradiol is solubilized in a solution comprising one or more solubilizers. A 28-day or monthly regimen of tablets or capsules can be packaged in a single blister pack having delivery days identified to improve compliance. Various example formulations of natural hormones, and the use of these formulations for hormone replacement therapies, each in accordance with the invention are set forth below.

Thus, in illustrative embodiments, the invention comprises a pharmaceutical formulation for administering estradiol and progesterone to a mammal in need thereof, comprising (i) solubilized estradiol and (ii) fully solubilized progesterone or partially solubilized progesterone in an oil wherein the oil comprises a medium chain fatty acid glycol ester or mixtures thereof. In certain such embodiments, the oil comprises medium chain fatty acid esters of glycerol, polyethylene glycol, or propylene glycol, or mixtures thereof and wherein the medium chain fatty acids are predominantly: C6 to C12 fatty acids, C6 to C10 fatty acids, C8 to C12 fatty acids, or C8 to C10 fatty acids, including saturated fatty acids. Certain such embodiments further comprise surfactants, including non-ionic surfactants. In certain embodiments, the progesterone is in micronized and solubilized forms, i.e., some of it is micronized and suspended and some of it is solubilized, in some cases to the extent of about 80% solubilized. Methods of use are also within the scope of this invention including a method for effecting hormone replacement therapy.

### BRIEF DESCRIPTION OF THE DRAWINGS/FIGURES

The accompanying drawings, which are incorporated herein and form a part of the specification, illustrate the present disclosure and, together with the description, further serve to explain the principles of the disclosure and to enable a person skilled in the pertinent art to make and use the disclosed embodiments.

FIG. 1 illustrates an exemplary manufacturing process of a fill material in accordance with various embodiments of the invention.

FIG. 2 illustrates an exemplary manufacturing process of a softgel material in accordance with various embodiments of the invention.

FIG. 3 illustrates an exemplary manufacturing process in accordance with various embodiments of the invention.

FIG. 4 illustrates a graph of the particle distribution obtained in Example 10.

FIG. 5 illustrates a dissolution study of a formulation in accordance with various embodiments of the invention.

### DETAILED DESCRIPTION OF THE ILLUSTRATED EMBODIMENTS

Frequently, higher recommended oral dosages of pharmaceuticals are necessary to treat a given disease state because many active ingredients are not completely absorbed by a patient in need of treatment. In other words, a better-absorbed dosage form of a medicament such as, for example, progesterone, or dosage forms that provide greater consistency of absorption of progesterone among subjects, alone or in combination with estradiol, may be able to be administered at dosage strengths lower than presently recommended, potentially resulting in a reduced or minimized side effect profile, among other potential benefits.

### DEFINITIONS

The term “micronized progesterone,” as used herein, includes micronized progesterone having an X50 particle size value below about 15 microns and/or having an X90 particle size value below about 25 microns.

The term “X50,” as used herein, means that one-half of the particles in a sample are smaller in diameter than a given number. For example, micronized progesterone having an X50 of 5 microns means that, for a given sample of micronized progesterone, one-half of the particles have a diameter of less than 5 microns. Similarly, the term “X90” means that ninety percent (90%) of the particles in a sample are smaller in diameter than a given number.

The term “medium chain,” as used herein, means any medium chain carbon-containing substance, including C4-C18, and including C6-C12 substances, fatty acid esters of glycerol, fatty acids, and mono-, di-, and tri-glycerides of such substances. For further illustration, C6-C14 fatty acids, C6-C12 fatty acids, and C8-C10 fatty acids are all medium chain fatty acids and may be used in instances in which this specification calls for use of medium chain fatty acids, e.g., medium chain fatty acid esters of glycerol or other glycols.

The term “uniform distribution” means at least one of uniform dispersion, solubility, or lack of agglomeration of progesterone in a dissolution test compared to PROMETRIUM at a similar dosage strength and the same USP dissolution apparatus.

The term “bioavailability,” as used herein, means the concentration of an active ingredient (e.g., progesterone or estradiol) in the blood (serum or plasma). The relative bioavailability may be measured as the concentration in the blood (serum or plasma) versus time. Other pharmacokinetic (PK) indicators may be used to measure and assess bioavailability, determined by suitable metrics including AUC, Cmax, and optionally, Tmax.

The term “AUC,” as used herein, refers to the area under the curve that represents changes in blood concentration of progesterone or estradiol (which is also referred to in the literature as 17β-estradiol, oestradiol, or E2) over time.

The term “Cmax,” as used herein, refers to the maximum value of blood concentration shown on the curve that represents changes in blood concentrations of progesterone or estradiol over time.

The term “Tmax,” as used herein, refers to the time that it takes for progesterone or estradiol blood concentration to reach the maximum value.

Collectively AUC, Cmax and, optionally, Tmax are the principle pharmacokinetic parameters that can characterize the pharmacokinetic response of a particular drug product such as progesterone in an animal, especially a mammal, including human, subject.

The term “solubilizer,” as used herein, means any substance or mixture of substances that may be used to solubilize or to enhance the solubility of an active pharmaceutical ingredient(s) (“API”), such as estradiol and/or progesterone, including, for example and without limitation, appropriate pharmaceutically acceptable excipients, such as solvents, co-solvents, surfactants, emulsifiers, oils and carriers.

The term “excipients,” as used herein, refers to non-active pharmaceutical ingredient substances such as carriers, solvents, oils, lubricants and others used in formulating pharmaceutical products. As used herein, active pharmaceutical ingredient is also referred to as “API.” They are generally safe for administering to animals, especially mammals, including humans, according to established governmental standards, including those promulgated by the United States Food and Drug Administration.

The term “oil,” as used herein, may be any pharmaceutically acceptable substance, such as an organic oil other than peanut oil, that would suspend and/or solubilize any suitable progesterone, starting material, or precursor, including micronized progesterone as described herein. More specifically, oils may include, for example and without limitation, medium chain fatty acids, generally of the group known as medium chain fatty acids consisting of at least one mono-, di-, and triglyceride, or derivatives thereof, or combinations thereof.

The term “fully solubilized progesterone,” as used herein, means progesterone which is about 100% in solution, e.g., at least 98% in solution.

The term “partially solubilized progesterone,” as used herein, means progesterone which is in any state of solubilization up to but not including about 100%, e.g., up to but not including 98% progesterone in solution and greater than or equal to 2% micronized progesterone in suspension.

As used herein, unless specified, estradiol includes estradiol in anhydrous and hemihydrate forms. In various embodiments, estradiol is solubilized. Solubilized estradiol may include estradiol that is approximately: 50% soluble in a solubilizer; 60% soluble in a solubilizer; 70% soluble in a solubilizer, 80% soluble in a solubilizer; 90% soluble in a solubilizer; 93% soluble in a solubilizer; 95% soluble in a solubilizer; 97% soluble in a solubilizer; and 99% soluble in a solubilizer. Any percentage of insolubility, or less than 100% solubility, would result in a formulation having properties of a suspension and not purely a solution.

The term “capsule,” as used herein, refers to a generally safe, readily dissolvable enclosure for carrying certain pharmaceutical products, and includes hard or soft shell capsules, including hard shell and soft shell gelatin capsules.

The term “softgel,” includes soft shell capsules, including soft-gelatin capsules and soft vegetable-based capsules, and soft capsules made from other materials providing the composition of such soft capsules are compatible with the formulations of the various embodiments described herein. A softgel may comprise two primary phases: a gel or vegetable-based capsule and a fill material in the preparation of a pharmaceutical formulation as described herein.

### EMBODIMENTS OF THE INVENTION

Provided herein are the following formulations: solubilized estradiol without progesterone; micronized progesterone without estradiol; micronized progesterone with partially solubilized progesterone; solubilized estradiol with micronized progesterone; solubilized estradiol with micronized progesterone in combination with partially solubilized progesterone; and solubilized estradiol with solubilized progesterone. The underlying formulation concepts provided herein may be used with other natural or synthetic forms of estradiol and progesterone. Micronization specifications, aspects and embodiments are further defined herein.

Generally, the pharmaceutical formulations described herein are prepared and administered as filled capsules, typically soft capsules of one or more materials well known in the art including, for example and without limitation, soft gelatin capsules. Micronized progesterone, as described herein, may also be prepared for administration in tablets or other well-known orally administered dosage forms using standard techniques.

Another aspect of the present disclosure includes a pharmaceutical formulation of micronized progesterone, micronized progesterone with partially solubilized progesterone and fully solubilized progesterone, wherein said formulation may provide increased progesterone bioavailability in a treated subject compared to the bioavailability provided by PROMETRIUM when administered at equal dosage strengths.

Estradiol may be obtained in any commercially available form, including crystalline, polymorph, hydrate and hemihydrates. Such forms can be micronized or non-micronized. Micronized estradiol may be selected from particle sizes with an average ranging from about 0.5 to about 50 μm. In various embodiments, a micronized estradiol hemihydrate is used.

In accordance with various aspects and embodiments, the solubility proportion (i.e., the proportion of a solute that enters solution) is notable. The weight ratio of estradiol to the weight of the entire solution is also notable due to the intended dose amounts, discussed herein. In particular, it is desirable to obtain a target dosage of estradiol in an amount of solution that may be readily administered via a capsule. For example, if it is desired to have a dose of estradiol in a capsule of between about 0.125 mg to about 2 mg, it would also be desirable to have a total solution weight to be between about 250 mg to about 400 mg, preferably about 300 mg to about 350 mg and more preferably about 325 mg. In various embodiments, the weight ratio of estradiol to total solution is from about 0.125 mg/50 mg to about 0.125 mg/1000 mg; or from about 1 mg/500 mg to about 1 mg/50 mg; or from about 1 mg/250 mg to about 1 mg/60 mg; or from about 1 mg/100 mg to about 1 mg/66 mg; or from about 2 mg/50 mg to about 2 mg/1000 mg. In various embodiments, the target fill weight for a single dose product is 325 mg, and a target fill weight for a combination product (e.g., two or more sterol APIs) is 650 mg.

Estradiol may be partially or substantially solubilized, including as follows: 50% soluble in a solubilizer; 60% soluble in a solubilizer; 70% soluble in a solubilizer, 80% soluble in a solubilizer; 90% soluble in a solubilizer; 93% soluble in a solubilizer; 95% soluble in a solubilizer; 97% soluble in a solubilizer; and 99% soluble in a solubilizer. Any percentage of insolubility, or less than 100% solubility, would result in a formulation having properties of a suspension and not purely a solution.

In various embodiments, estradiol is completely solubilized. As presented herein, higher solubility of estradiol in the present formulations enhances the various aspects, embodiments, and improvements taught herein. Solubility may be expressed as a mass fraction (% w/w or mg/g).

In illustrative embodiments, total progesterone, i.e., dissolved and micronized, is 20 to 50 wt %, e.g., 30 to 35 wt %; estradiol is 0.1 to 0.8 wt %, e.g., 0.15 to 0.35 wt %.

Other aspects of the present disclosure further provide: more uniform dissolution of progesterone, and reduced intra- and inter-patient blood level variability in formulations of progesterone of the present disclosure, typically in combinations with solubilized estradiol, when compared to equal dosages of PROMETRIUM). Blood level variability is also compared at equal sampling times following administration. Not to be limited by theory, these aspects are believed to be influenced by the percentage of solubilized progesterone in a respective formulation wherein such more uniform dissolution of progesterone, and lower intra- and inter-patient blood level variability, are influenced by a greater proportion of solubilized progesterone relative to total progesterone. A reduced food effect with the present formulations comprising progesterone may also be implicated.

According to the PROMETRIUM prescribing information, clinical trials have shown significant patient variability. For example, a clinical trial involving post-menopausal women who were administered PROMETRIUM once a day for five days resulted in the mean PK parameters listed in the following table:

| | PROMETRIUM Capsules Daily Dose | | |
|---|---|---|---|
| Parameter | 100 mg | 200 mg | 300 mg |
| Cₘₐₓ | 17.3 +/− 21.9 | 38.1 +/− 37.8 | 60.6 +/− 72.5 |
| (ng/ml) | | | |
| Tₘₐₓ | 1.5 +/− 0.8 | 2.3 +/− 1.4 | 1.7 +/− 0.6 |
| (hr) | | | |
| AUC₀₋₁₀ | 43.4 +/− 30.8 | 101.2 +/− 66.0 | 175.7 +/− 170.3 |
| (ng × hr/ml) | | | |

In particular illustrative aspects and embodiments of this invention, it is possible, though not necessary, to reduce the standard deviations in one or more of these PK parameters.

More uniform dissolution of progesterone in a formulation of the present disclosure compared to the dissolution of PROMETRIUM at equal dosage strengths and using the same USP apparatus can be determined using standard techniques established for API dissolution testing, including that which is described in the examples below.

Reduced intra- and inter-patient variability of progesterone formulated pursuant to the present disclosure compared to PROMETRIUM can be demonstrated via a fed bio-study such as that described below.

Other aspects of the present disclosure includes the use of formulations as described herein wherein progesterone is at least one API in said formulation for the treatment of an animal, especially a mammal, including humans: for endometrial hyperplasia; for secondary amenorrhea; as a method of treatment for preterm birth, when said animal has a shortened cervix, and other disease states or conditions treated with supplemental progesterone (collectively, “Progesterone-deficient States”); and the use of formulations as described herein wherein estradiol is at least one API in said formulation for the treatment of an animal, especially a mammal, including humans, having menopause-related symptoms including, for example, vasomotor symptoms; in relation to treatment of hypoestrogenism related symptoms including, for example and without limitation, hot flashes and night sweats (vasomotor symptoms), sleep disturbances, mood changes and vulvo-vaginal atrophy; and osteoporosis and other non-menopausal disease states or conditions treated with supplemental estrogen (collectively, “Estrogen-deficient states”), each in a subject in need of treatment, and each with a non-toxic effective amount of said formulations. As used herein, the term “treatment”, or a derivative thereof, contemplates partial or complete inhibition of the stated disease state when a formulation as described herein is administered prophylactically or following the onset of the disease state for which such formulation is administered. For the purposes of the present disclosure, “prophylaxis” refers to administration of the active ingredient(s) to an animal, especially a mammal, to protect the animal from any of the disorders set forth herein, as well as others.

Unless otherwise specified, “natural,” as used herein with reference to hormones discussed herein, means bio-identical hormones formulated to match the chemical structure and effect of those that occur naturally in the human body (endogenous). An exemplary natural estrogen is estradiol (also described as 17β-estradiol and E2) and a natural progestin is progesterone. An exemplary cyclic-sequential regimen comprises delivery of from about 0.125 mg to about 2.0 mg of estradiol daily for 14-18 days, followed by delivery of from about 0.125 mg to about 2 mg of estradiol and about 25 mg to about 200 mg of progesterone daily for 10-14 days. Cyclic-sequential regimens may be especially useful for menopausal females. Other exemplary dosage strengths for estradiol for use in the formulations described herein include, without limitation, 0.125, 0.25, 0.375, 0.50, 0.625, 0.75, 1.00, 1.125, 1.25, 1.375, 1.50, 1.625, 1.75 and 2.00 mg. Other exemplary dosage strengths for progesterone for use in the formulations described herein include, without limitation, 25, 50, 75, 100, 125, 150, 175, 200, 250, 300, 350 and 400 mg. These dosage strengths for each of estradiol and progesterone can be administered in formulations described herein either alone or in combination.

Progesterone active pharmaceutical ingredient may be micronized via any one of the multiple methods typically utilized by the ordinarily skilled artisan. In various embodiments, micronized progesterone has an X50 particle size value of less than about 15 microns, less than about 10 microns, less than about 5 microns and/or less than about 3 microns. In various embodiments, micronized progesterone has an X90 particle size value of less than about 25 microns, less than about 20 microns, and/or less than about 15 microns.

Particle size may be determined in any suitable manner. For example, a Beckman Coulter LS 13 320 Laser Diffraction Particle Size Analyzer (the “Beckman Device”) may be used to determine particle size. As described above, particle size may be represented by various metrics, for example, through an X50 particle size, and/or X90 particle size, or similar descriptions of particle size.

The Beckman Device may be used with various modules for introducing a sample for analysis. The Beckman Device may be used with the LS 13 320 Universal Liquid Module (“ULM”). The ULM is capable of suspending samples in the size range of 0.017 μm to 2000 μm. The ULM is a liquid based module that allows for delivery of the sample to the sensing zone. The ULM recirculates the sample through the Beckman Device. The ULM comprises two hoses, one for fluid delivery and another for waste. The total volume used may be 125 mL or less. A sample mass of from about 1 mg to about 10 g may be used. The ULM may interact with the Beckman Device via pins that fit into slots on the ULM. The ULM may use a variety of suspension fluids, for example, water, butonol, ethanol, chloroform, heptanes, toluene, propanol, COULTER Type 1B Dispersant (“Coulter 1B”), and a variety of other suspension fluids. Surfactants may also be used, though pump speed should be adjusted to prevent excessive bubbling. Coulter 1B may comprise one or more of acetaldehyde, ethylene oxide, and/or 1,4-dioxane. The Beckman Device may be configured to use a variety of optical theories, including the Fraunhofer optical model and the Mie Theory.

The Beckman Device may comprise software to control the Beckman Device while the ULM is in use. The software may control, for example, pump speed, use of de-bubble routine, rinse routine, sonicate routine, and fill routine, among others. Parameters regarding the sample run may also be configured. For example, run length may be set. Though any suitable run length may be used, in various embodiments, a time period of 30 seconds to 120 seconds, and preferably between 30 seconds and 90 seconds may be used.

The Beckman Device may be used with the LS 13 320 Micro Liquid Module (“MLM”). The MLM is capable of suspending samples in the size range of 0.4 μm to 2000 μm. The MLM is a liquid based module that allows for delivery of the sample to the sensing zone. The MLM includes a stirrer. The total volume used may be 12 mL or less. The MLM may use a variety of suspension fluids, both aqueous and non-aqueous.

Each of estradiol and progesterone as described herein can be formulated alone pursuant to the teachings below. These formulations can be prepared for oral administration or can be combined, based on compatibility, for co-administration of estradiol and progesterone in a single oral unit dosage form.

Progesterone formulations and estradiol formulations of the present disclosure are prepared via blending with a pharmaceutically acceptable oil. Generally, the oil comprises at least one medium chain fatty acid such as medium chain fatty acids consisting of at least one mono-, di-, or triglyceride, or derivatives thereof, or combinations thereof. Optionally added are other excipients including, for example and without limitation, anti-oxidants, lubricants and the like. Sufficient oil is used to form a suspension of micronized progesterone or, in the alternative, to solubilize progesterone.

Pharmaceutically acceptable oils include, without limitation, caproic acid; caprylic acid; capric acid; lauric acid; myristic acid; and linoleic acid. Pharmaceutically acceptable oils include mono-, di-, or triglycerides and of such acids, and combinations. Pharmaceutically acceptable oils can include succinic acid, glycerin, propylene glycol, and a polyethylene glycol. Pharmaceutically acceptable oils also include esters of saturated coconut and palm kernel oil and derivatives thereof; triglycerides of fractionated vegetable fatty acids; and combinations thereof. Pharmaceutically acceptable oils also include caprylic/capric triglycerides (e.g., MIGLYOL 810, 812, 816 or 829, SASOL Germany GMBH, Hamburg); caproic/caprylic/capric/lauric triglycerides; caprylic/capric/linoleic triglycerides; and caprylic/capric/succinic triglycerides. Pharmaceutically acceptable oils also include medium chain mono- and di-glycerides (e.g., CAPMUL MCM). Pharmaceutically acceptable oils also include propylene glycol monocaprylate; propylene glycol monocaprate (e.g., CAPMUL PG-8, ABITEC, Columbus Ohio); propylene glycol dicaprylate; and propylene glycol dicaprate. Pharmaceutically acceptable oils also include a diethylene glycol monoether (including 2-(2-ethoxyethoxy)ethanol (TRANSCUTOL)).

In other aspects and embodiments, progesterone and estradiol are fully solubilized using, for example and without limitation, sufficient amounts of: TRANSCUTOL and MIGLYOL; TRANSCUTOL, MIGLYOL and CAPMUL PG 8 and/or PG 10; CAPMUL MCM; CAPMUL and a non-ionic surfactant; and CAPMUL MCM and GELUCIRE.

Various ratios of these oils can be used for full solubilization of progesterone and estradiol. CAPMUL MCM and a non-ionic surfactant, e.g., GELUCIRE 44/14 (lauroyl macrogol-32 glycerides EP; lauroyl polyoxyl-32 glycerides NF; lauroyl polyoxylglycerides (USA FDA IIG)), can be used at ratios of about 99:1 to 2:1, including, for example and without limitation: 60:40, 65:35, 70:30, 75:25, 80:10, 80:15, 85:20, 90:10, and 98:1. The ratios of oil (e.g., medium chain fatty acid esters of monoglycerides and diglycerides) to non-ionic surfactant can be significantly higher. For example, in certain examples, below, CAPMUL MCM and GELUCIRE were used in ratios of up to about 65:1, e.g., 8:1, 22:1, 49:1, 65:1 and 66:1. See, e.g., Tables 13-17, below. Thus, useful ratios can be 8:1 or greater, e.g., 60 to 70:1. Among other combinations, these oils and/or solubilizers, as defined herein, and combinations thereof, can be used to form combination estradiol and progesterone formulations of the present disclosure.

Combinations of these oils can produce partially solubilized progesterone, depending upon the desired unit dosage amount of progesterone. The greater the amount of progesterone per unit dosage form, the less progesterone may be solubilized. The upward limit of dosage strength per unit dose it generally limited only by the practical size of the final dosage form.

In illustrative embodiments of the invention, oils used to solubilize estradiol and to suspend, partially solubilize, or fully solubilize progesterone include medium chain fatty acid esters, (e.g., esters of glycerol, polyethylene glycol, or propylene glycol) and mixtures thereof. In illustrative embodiments, the medium chain fatty acids are C6 to C14 or C6 to C12 fatty acids. In illustrative embodiments, the medium chain fatty acids are saturated, or predominantly saturated, e.g., greater than about 60% or greater than about 75% saturated. In illustrative embodiments, estradiol or progesterone (or both) are soluble in the oils at room temperature, although it may be desirable to warm the oils up until they are in a liquid state. In illustrative embodiments, the oil or oil/surfactant is liquid at between room temperature and about 50° C., e.g., at or below 50° C., at or below 40° C., or at or below 50° C. In illustrative embodiments, GELUCIRE 44/14 is heated to about 65° C. and CAPMUL MCM is heated to about 40° C. to facilitate mixing of the oil and non-surfactant, although such heating is not necessary to dissolve the estradiol or progesterone. In illustrative embodiments, the solubility of estradiol in the oil (or oil/surfactant) is at least about 0.5 wt %, e.g., 0.8 wt % or higher, or 1.0 wt % or higher. However, much higher solubility can be achieved. For example, as shown in Example 5, below, estradiol is stable in solution in CAPMUL MCM at 12 mg/g (which is approximately equal to 12 mg/ml). As shown in Example 17, such solubility is favored over results observed in longer chain and unsaturated fatty acid esters.

Illustrative examples of mono- and diglycerides of medium chain fatty acids include, among others, CAPMUL MCM, CAPMUL MCM C10, CAPMUL MCM C8, and CAPMUL MCM C8 EP. These oils are C8 and C10 fatty acid mono- and diglycerides. Illustrative examples of oils that are triglycerides of medium chain fatty acids include, among others, MIGLYOL 810 and MIGLYOL 812.

Illustrative examples of oils that are medium chain fatty acid esters of propylene glycol include, among others, CAPMUL PG-8, Capmul PG-2L EP/NF, CAPMUL PG-8 NF, Capmul PG-12 EP/NF and Capryol. Other illustrative examples include MIGLYOL 840.

Illustrative examples of oils that are medium chain fatty acid esters of polyethylene glycol include, among others, GELUCIRE 44/14 (PEG-32 glyceryl laurate EP), which is polyethylene glycol glycerides composed of mono-, di- and triglycerides and mono- and diesters of polyethylene glycol. Without intending to be bound to any particular mechanism, it appears that at least in formulations comprising small amounts of GELUCIRE, e.g., 10 wt % or less, the primary function of this oil is as a non-ionic surfactant.

These illustrative examples comprise predominantly medium chain length, saturated, fatty acids, specifically predominantly C8 to C12 saturated fatty acids.

It will be understood that commercially available fatty acid esters of glycerol and other glycols are often prepared from natural oils and therefore may comprise components additional to the fatty acid esters that comprise the predominant (by weight) component(s) and that therefore are used to characterize the product. Such other components may be, e.g., other fatty acid triglycerides, mono- and diesters, free glycerol, or free fatty acids. So, for example, when an oil/solubilizing agent is described herein as a saturated C8 fatty acid mono- or diester of glycerol, it will be understood that the predominant component of the oil, i.e., >50 wt % (e.g., >75 wt %, >85 wt % or >90 wt %) are caprylic monoglycerides and caprylic diglycerides. For example, the Technical Data Sheet by ABITEC for CAPMUL MCM C8 describes CAPMUL MCM C8 as being composed of mono and diglycerides of medium chain fatty acids (mainly caprylic) and describes the alkyl content as <=1% C6, >=95% C8, <=5% C10, and <=1.5% C12 and higher.

By way of further example, MIGLYOL 812 is generally described as a C8-C10 triglyceride because the fatty acid composition is at least about 80% caprylic (C8) acid and capric (C10) acid. However, it can also comprise small amounts of other fatty acids, e.g., less than about 5% of caproic (C6) acid, lauric (C12) acid, and myristic (C14) acid.

Specifically, a product information sheet for MIGLYOL by SASOL provides the composition of fatty acids as follows:

| Tests | 810 | 812 | 818 | 829 | 840 |
|---|---|---|---|---|---|
| Caproic acid | max. 2.0 | max. 2.0 | max. 2 | max. 2 | max. 2 |
| (C6:0) | | | | | |
| Caprylic acid | 65.0-80.0 | 50.0-65.0 | 45-65 | 45-55 | 65-80 |
| (C8:0) | | | | | |
| Capric acid | 20.0-35.0 | 30.0-45.0 | 30-45 | 30-40 | 20-35 |
| (C10:0) | | | | | |
| Lauric acid | max. 2 | max. 2 | max. 3 | max. 3 | max. 2 |
| (C12:0) | | | | | |
| Myristic acid | max. 1.0 | max. 1.0 | max. 1 | max. 1 | max. 1 |
| (C14:0) | | | | | |
| Linoleic acid | — | — | 2-5 | — | — |
| (C18:2) | | | | | |
| Succinic acid | — | — | — | 15-20 | — |

Where certain embodiments of this invention are described as comprising (or consisting essentially of) a capsule shell, estradiol solubilized in C8-C10 triglycerides, and a thickening agent, it will be understood that the fatty acid esters component of the formulation may be, e.g., MIGLYOL 812 or a similar product.

By way of further illustration, GELUCIRE 44/14 is generally described as lauroyl polyoxyl-32 glycerides, i.e., polyoxyethylene 32 lauric glycerides (which is a mixture of mono-, di-, and triesters of glycerol and mono- and diesters of PEGs) because the fatty acid composition is 30 to 50% lauric acid and smaller amounts of other fatty acids, e.g., up to 15% caprylic acid, up to 12% capric acid, up to 25% myristic acid, up to 25% palmitic acid, and up to 35% stearic acid. The product may also contain small amounts of non-esterified glycols. Where certain embodiments of this invention are described as comprising (or consisting essentially of) a capsule shell, estradiol solubilized in triglycerides, and a thickening agent that is a non-ionic surfactant comprising C8 to C18 fatty acid esters of glycerol and polyethylene glycol, it will be understood that the thickening agent component of the formulation may be, e.g., GELUCIRE 44/14 or a similar product.

Similarly, where certain embodiments of this invention are described as comprising (or consisting essentially of) a capsule shell, estradiol solubilized in triglycerides, and a thickening agent that is a non-ionic surfactant comprising PEG-6 stearate, ethylene glycol palmitostearate, and PEG-32 stearate, it will be understood that the thickening agent component of the formulation may be, e.g., Tefose 63 or a similar product.

Mixtures of medium chain fatty acid glycerides, e.g., C6-C12, C8-C12, or C8-C10 fatty acid mono- and diglycerides or mono-, di-, and triglycerides are very well suited for dissolving estradiol. Good results have been obtained with an oil that is predominantly a mixture of C8-C10 saturated fatty acid mono- and diglycerides. Longer chain glycerides appear to be not as well suited for dissolution of estradiol. On the other hand, high solubility of progesterone has been obtained in mixtures that are predominantly medium chain fatty acid triglycerides.

High solubility of estradiol has been obtained in 2-(2-Ethoxyethoxy)ethanol, e.g., TRANSCUTOL and in Propylene glycol monocaprylate, e.g., Capryol™ 90 (Gattefosse).

In illustrative embodiments of the invention, the selected oil does not require excessive heating in order to solubilize progesterone or estradiol. For example, when the formulation comprises medium chain fatty acid mono- and diglycerides (e.g., CAPMUL MCM) and polyethylene glycol glycerides (e.g., GELUCIRE) as a surfactant, the oil and/or the surfactant can be warmed up, e.g., to about 65° C. in the case of the surfactant and less in the case of the oil, to facilitate mixing of the oil and surfactant. The estradiol can be added at this temperature or at lower temperatures as the mixture cools or even after it has cooled as temperatures above room temperature, e.g., about 20 C, are not required to solubilize the estradiol in preferred oils. The progesterone can also be added as the mixture cools, e.g., to below about 40° C. or to below about 30 C, even down to room temperature.

In addition to the oils referenced above, other solubilizers include, for example and without limitation, glyceryl mono- and di-caprylates, propylene glycol and 1,2,3-propanetriol (glycerol, glycerin, glycerine).

Anionic and/or non-ionic surfactants can be used in other embodiments of the presently disclosed formulations containing estradiol, progesterone or a combination thereof.

In certain embodiments, a non-ionic surfactant is used. Exemplary non-ionic surfactants may include, for example and without limitation, one or more of oleic acid, linoleic acid, palmitic acid, and stearic acid esters or alcohols. In further embodiments, the non-ionic surfactant may comprise polyethylene sorbitol esters, including polysorbate 80, which is commercially available under the trademark TWEEN 80® (Sigma Aldrich, St. Louis, Mo.). Polysorbate 80 comprises approximately 60%-70% oleic acid with the remainder comprising primarily linoleic acids, palmitic acids, and stearic acids. Polysorbate 80 may be used in amounts ranging from about 5 to 50%, and in certain embodiments, about 30% of the formulation total mass.

In further various embodiments a surfactant (e.g., a non-ionic surfactant) is included.

In various embodiments where CAPMUL MCM and polysorbate 80 are present, CAPMUL MCM may be used in amounts greater than about 15% of the formulation's total mass. Polysorbate 80 may be used in amounts ranging from about 5 to 50%, and in certain embodiments, about 30% of the formulation total mass.

In various other embodiments, the non-ionic surfactant is selected from one or more of glycerol and polyethylene glycol esters of fatty acids, for example, lauroyl macrogol-32 glycerides and/or lauroyl polyoxyl-32 glycerides, commercially available as GELUCIRE, including, for example, GELUCIRE 44/11 and GELUCIRE 44/14. These surfactants may be used at concentrations greater than about 0.01%, and typically in various amounts of about 0.01%-10.0%, 10.1%-20%, and 20.1%-30%. In certain examples, below, GELUCIRE 44/14 is used as a surfactant in amounts of 1 to 10 wt %. See, e.g., Tables 13-17, below. Other non-ionic surfactants include, e.g., Labrasol® PEG-8 Caprylic/Capric Glycerides (Gattefosse) and Labarafil® corn/apricot oil PEG-6 esters (Gattefosse).

In other embodiments, a lubricant is used. Any suitable lubricant may be used, such as for example lecithin. Lecithin may comprise a mixture of phospholipids. Suitable lubricants may also comprise calcium stearate, ethyl oleate, ethyl laurate, glycerin, glyceryl palmitostearate, hydrogenated vegetable oil, magnesium oxide, magnesium stearate, mannitol, poloxamer, glycols, and phospholipid mixtures.

In additional embodiments, an antioxidant is used. Any suitable antioxidant may be used such as, for example and without limitation, butylated hydroxytoluene.

For example, in various embodiments, a pharmaceutical formulation comprises about 20% to about 80% carrier by weight, about 0.1% to about 5% lubricant by weight, and about 0.01% to about 0.1% antioxidant by weight.

The choice of excipient will, to a large extent, depend on factors such as the particular mode of administration, the effect of the excipient on solubility and stability, and the nature of the dosage form. Excipients used in various embodiments may include colorants, flavoring agents, preservatives and taste-masking agents. Colorants, for example, may comprise about 0.1% to about 2% by weight. Preservatives may comprise methyl and propyl paraben, for example, in a ratio of about 10:1, and at a proportion of about 0.005% and 0.05% by weight.

As is with all oils, solubilizers, excipients and any other additives used in the formulations described herein, each is to be non-toxic and pharmaceutically acceptable.

As referenced above, the formulations of the present disclosure can be orally administered, typically via, for example, capsules such as soft capsules. Oral administration may involve swallowing, so that the compound enters the gastrointestinal tract, or buccal or sublingual administration may be employed by which the compound enters the blood stream directly from the mouth.

Solubilized estradiol in accordance with various embodiments may be formulated as a vaginal suppository or vaginal cream for administration onto the vulva or into the vagina, cervix, or uterus of a human. Capsules (including softgel capsules) containing solubilized estradiols also may be administered vaginally, including insertion of a capsule (e.g., a softgel) directly into the vaginal cavity or delivery of such capsule contents into the vaginal cavity.

Solubilized estradiol in accordance with various embodiments may be formulated for intraperitoneal administration and atomization, such as nasal mist administration, using standard techniques well known in the art.

In accordance with various embodiments, formulations do not include peanut oil. The lack of peanut oil obviates the risk posed to those having peanut-based allergies.

Thus, an illustrative embodiment of a pharmaceutical composition of the invention comprises solubilized estradiol, progesterone at least 75% of the progesterone being solubilized (the balance being micronized as discussed elsewhere herein), and an oil, wherein the oil is medium chain fatty acid mono- and diesters of glycerol, with or without surfactant. In certain embodiments, a specification for progesterone is set at >80% solubilized, <20% micronized or >85% solubilized, <15% micronized. Specific examples of such illustrative embodiments, with GELUCIRE as surfactant, in which at least about 85% of the progesterone can be solubilized, include, e.g., the following four formulations:

| | | |
|---|---|---|
| Ingredient(s) | Amount (% w/w) | Qty/Capsule (mg) |

Formulation A- P: 50/E2: 0.25:

| | | |
|---|---|---|
| Progesterone, USP, micronized | 33.33 | 50.00 |
| Estradiol Hemihydrate | 0.17 | 0.26 |
| CAPMUL MCM, NF | 65.49 | 98.24 |
| GELUCIRE 44/14, NF | 1.00 | 1.50 |
| Total | 100.00 | 150.00 |

Formulation B- P: 50/E2: 0.5:

| | | |
|---|---|---|
| Progesterone, USP, micronized | 33.33 | 50.00 |
| Estradiol Hemihydrate | 0.35 | 0.52 |
| CAPMUL MCM, NF | 65.32 | 97.98 |
| GELUCIRE 44/14, NF | 1.00 | 1.50 |
| Total | 100.00 | 150.00 |

Formulation C - P: 100/E2: 0.5:

| | | |
|---|---|---|
| Progesterone, USP, micronized | 33.33 | 100.00 |
| Estradiol Hemihydrate | 0.17 | 0.52 |
| CAPMUL MCM, NF | 65.49 | 196.48 |
| GELUCIRE 44/14, NF | 1.00 | 3.00 |
| Total | 100.00 | 300.00 |

Formulation D - P: 100/E2: 1:

| | | |
|---|---|---|
| Progesterone, USP, micronized | 33.33 | 100.00 |
| Estradiol Hemihydrate | 0.34 | 1.03 |
| CAPMUL MCM, NF | 65.32 | 195.97 |
| GELUCIRE 44/14, NF | 1.00 | 3.00 |
| Total | 100.00 | 300.00 |

Formulation E- P: 200/E2: 2:

| | | |
|---|---|---|
| Progesterone, USP, micronized | 33.33 | 200.00 |
| Estradiol Hemihydrate | 0.34 | 2.06 |
| CAPMUL MCM, NF | 65.32 | 391.94 |
| GELUCIRE 44/14, NF | 1.00 | 6.00 |
| Total | 100.00 | 600.00 |

*Note:
1.00 mg Estradiol equivalent to 1.03 mg Estradiol Hemihydrate.

Illustrative embodiments in which the carrier is a medium fatty acid ester of a glycol and which comprise a non-ionic surfactant as described herein are in liquid form, i.e., not gels, hard fats or other solid forms.

In general terms, the above formulations comprise 30 to 35 wt % progesterone, 0.1 to 0.4 wt % estradiol (or estradiol hemihydrate), 55 to 75 wt % of an oil that is predominantly medium chain fatty acid mono- and diglycerides, such as CAPMUL MCM, and 0.5 to 10 wt % non-ionic surfactant, such as GELUCIRE 44/14. The above formulations may be modified to comprise excipients, e.g., gelatin such as Gelatin 200 Bloom, glycerin, coloring agents such as Opatint red and white, and, optionally, MIGLYOL 812.

Estradiol solubilization helps ensure high content uniformity and enhanced stability.

Fully solubilized progesterone formulations or partially solubilized progesterone formulations in which at least about 50% of the progesterone, e.g., 75%, 80%, 85%, 90%, or >95%, is solubilized appear to provide improved PK-related properties.

Pharmaceutical formulations in accordance with various embodiments comprise solubilized estradiol, which comprises estradiol and a solubilizer. In further embodiments, a pharmaceutical formulation comprises solubilized estradiol, a solubilizer, and a lubricant. In still further embodiments a pharmaceutical formulation comprises solubilized estradiol, a solubilizer, a lubricant, and optionally an antioxidant. In still further embodiments, a pharmaceutical formulation comprises solubilized estradiol, a medium chain triglyceride, and monoglycerides/diglycerides/triglycerides of caprylic/capric acid, and a non-ionic surfactant In still further embodiments, a pharmaceutical formulation may comprise solubilized estradiol, a medium chain triglyceride, and a diethylene glycol monoethylether. In still further embodiments, a pharmaceutical formulation may comprise solubilized estradiol, medium chain triglyceride, or one or more monoglycerides/diglycerides/triglycerides of caprylic/capric acid, and polysorbate 80. Various further embodiments also comprise lecithin and optionally butylated hydroxytoluene.

In additional embodiments a pharmaceutical formulation comprises solubilized estradiol, and optionally one or more of a carrier or solvent, a lubricant, an antioxidant, and other pharmaceutically acceptable excipients.

Formulations in accordance with various embodiments may be administered alone or combination with one or more other drugs {or as any combination thereof).

In various embodiments, solubilized estradiol is administered in a capsule. Capsules (made, for example, from gelatin or hydroxypropylmethylcellulose), may be arranged in bottles, blisters or cartridges. A capsule can comprise two primary phases: a capsule (e.g., gel or vegetable-based) and a fill material. The capsule may define an inner volume that may contain the fill material. The fill material may comprise a liquid, such as an oil, a solution, a suspension, an emulsion, or the like.

Capsules may be prepared using one or more film forming polymers. Suitable film forming polymers include natural polymers, such as gelatin, and synthetic film forming polymers, such as modified celluloses. Suitable modified celluloses include, but are not limited to, hydroxypropyl methyl cellulose, methyl cellulose, hydroxypropyl methyl cellulose acetate succinate, hydroxypropyl methyl cellulose phthalate, and cellulose acetate phthalate.

Hard or soft shell capsules can be used to administer the API.

Hard shell capsules are typically prepared by forming the two capsule halves, filling one of the halves with the fill solution, and then sealing the capsule halves together to form the finished capsule.

Soft gelatin capsules are typically prepared using a rotary die encapsulation process.

Suitable shell additives, for either a hard or soft shell capsules, may include plasticizers, opacifiers, colorants, humectants, preservatives, flavorings, and buffering salts and acids, and solubilizers, including selected carriers and solvents, are selected in various embodiments to facilitate traversal of solubilized estradiol across a cell membrane. Mono/di/triglycerides are particularly suited to aid in cellular absorption. Thus, in the digestive system, formulations in accordance with various embodiments may be taken into the bloodstream for systemic circulation after absorption in the digestive system.

Plasticizers are chemical agents added to gelatin to make the material softer and more flexible. Suitable plasticizers include, but are not limited to, glycerin, sorbitol solutions which are mixtures of sorbitol and sorbitan, and other polyhydric alcohols such as propylene glycol and maltitol or combinations thereof.

Opacifiers are used to opacify the capsule shell when the encapsulated active agents are light sensitive. Suitable opacifiers include titanium dioxide, zinc oxide, calcium carbonate ami combinations thereof.

Colorants can be used to for marketing and product identification/differentiation purposes. Suitable colorants Include synthetic and natural dyes and combinations thereof.

Flavorings can be used to mask unpleasant odors and tastes of fill formulations. Suitable flavorings include synthetic and natural flavorings. The use of flavorings can be problematic due to the presence of aldehydes which can cross-link gelatin. As a result, buffering salts and acids can be used In conjunction with flavorings that contain aldehydes in order to inhibit cross-linking of the gelatin.

In accordance with various embodiments, a softgel dosage form is used.

Dissolution of the capsule may commence at various anatomical locations, such as along the digestive system (mouth, esophagus, stomach and intestines), or other body cavities such as the vaginal cavity.

As the capsule dissolves, the inner volume may come into fluid communication with the digestive system, allowing the fill material to leach outside the capsule. A capsule may also be punctured, cut, or otherwise opened outside a body. The fill material may then be poured or squeezed outside the softgel and applied on or in the body, such as within the vaginal cavity. The capsule can also be designed to dissolve within the vaginal cavity and be absorbed together with the fill material.

Humectants can be used to suppress the water activity of certain capsules, including softgels. Suitable humectants include glycerin and sorbitol, which are often components of the plasticizer composition. Due to the low water activity of dried, properly stored capsules, the greatest risk from microorganisms comes from molds and yeasts. For this reason, preservatives can be incorporated into the capsule shell. Suitable preservatives include alkyl esters of p-hydroxy benzoic acid such as methyl, ethyl, propyl, butyl and heptyl esters (collectively known as “parabens”) or combinations thereof.

According to various embodiments described herein, a 28-day or monthly regimen of capsules can be packaged in a single kit (e.g., a blister pack) having administration days identified to improve compliance and reduce associated symptoms, among others. One or more of the capsules may contain no estradiol, for example, and/or no progesterone. Capsules that comprise no estrogen or progesterone API may be referred to as placebos. A blister pack can have a plurality of scores or perforations separating blister pack into 28 days. Each day may further comprise a single blister or a plurality of blisters. In various embodiments, each unit dose may contain micronized and/or partially solubilized, or fully solubilized progesterone and/or solubilized estradiol in amounts as set forth herein above, although other dose ranges may be contemplated. In addition, kits having other configurations are also contemplated herein. For example, without limitation, kits having such blister packs may contain any number of capsules.

Orally administered formulations of the present disclosure containing micronized and/or partially solubilized, or fully solubilized, progesterone are also used for the treatment of endometrial hyperplasia, secondary amenorrhea and other disease states treated with supplemental progesterone. Generally, progesterone-containing formulations described herein are used to treat the effects of the administration of supplemental estrogen whether administered alone or in combination with solubilized estradiol of the present disclosure or other estrogen-containing formulations. In various other embodiments, a capsule containing formulations of the present disclosure, for example a softgel capsule, may be applied in or around the vagina.

Formulations of the present disclosure containing solubilized estradiol are used to treat Estrogen-deficient states, including vasomotor symptoms, for example, in relation to treatment of hypoestrogenism related symptoms including, for example and without limitation, hot flashes and night sweats (vasomotor symptoms), sleep disturbances, mood changes, vulvo-vaginal atrophy, and osteoporosis and other non-menopausal disease states treated with supplemental estrogen.

Formulations of the present disclosure containing solubilized estradiol may be used to treat or prevent atrophic vaginitis or vulvo-vaginal atrophy. In various embodiments, a capsule, for example a softgel capsule, may be applied in or around the vagina.

Formulations in accordance with various embodiments may be used to treat moderate to severe vasomotor symptoms associated with menopause. In various embodiments, a softgel capsule may be opened and the fill material applied in or around the vagina or inserted into the vagina and therein dissolve and be absorbed into the vaginal tissue. However, in various embodiments the softgel capsules are taken orally.

Additional objects of the present disclosure include: providing increased patient compliance secondary to ease of use; providing increased physician adoption secondary to ease of use/instruction with less worry of side effects from inappropriate usage; providing decreased side-effects from erroneous use (decreased irregular bleeding); providing better efficacy/control of symptoms secondary to appropriate use; reducing the metabolic and vascular side effects of the commonly used synthetic progestins when administered alone or in combination with an estrogen (norethindrone acetate, medroxyprogesterone acetate, etc.) including, for example, stroke, heart attacks, blood clots and breast cancer.

In accordance with various embodiments, enhanced bioavailability of estradiol is provided, such as over conventional estradiol formulations. While not bound by theory, it is believed that in conventional estradiol (including non-solubilized), a portion of the estradiol in a given dose is not completely or consistently absorbed by a human body. Formulations in accordance with various embodiments disclosed herein however, are more easily absorbed by a human body or are better able to achieve a therapeutic effect in vivo. Thus, a lower dosage strength of estradiol may be used in a given dose to achieve the same therapeutic effect. Bioavailability comparisons to commercially available forms, such as tablet forms, may be determined by standard techniques, such as pharmacokinetic analysis or clinical trials.

### EXAMPLES

### Example 1

### Estradiol Solubility

In various experiments, suitable solvents were determined for providing sufficient solubility to make 2 mg of estradiol in a 100 mg fill mass, with a desired goal of achieving ˜20 mg/g solubility for estradiol. Initial solubility experiments were done by mixing estradiol with various solvents, saturate the solution with the estradiol, equilibrate for at least 3 days and filter the un-dissolved particles and analyzing the clear supernatant for the amount of estradiol dissolved by HPLC.

Estradiol solubility experiments were performed. From this list at least one item (e.g. propylene glycol) is known to be unsuitable for encapsulation in more than 20% w/w concentration.

TABLE 1

| Ingredient | Solubility (mg/g) |
|---|---|
| PEG 400 | 105* |
| Propylene Glycol | 75* |
| Polysorbate 80 | 36* |
| TRANSCUTOL HP | 141 |
| CAPMUL PG8 | 31.2 |

*Literature reference -Salole, E. G. (1987) The Physicochemical Properties of Oestradiol, J Pharm and Biomed Analysis, 5, 635-640.

As shown in Table 1.1, the solubility of estradiol was at least 6 mg/gm in MIGLYOL TRANSCUTOL (81:19 to 95:5), in MIGLYOL; ethanol (91:11), and in MIGLYOL:CAPMUL PG8 (88:11), but not in MIGLYOL:TRANSCUTOL (96:4), MIGLYOL:Labrasol (70:30 to 80:20), or MIGLYOL:CAPMUL PG8 (86:14).

TABLE 1.1

| Composition | Estradiol Solubility >6 mg/g |
|---|---|
| Miglyol:Transcutol (81:19) | Yes |
| Miglyol:Transcutol (88:11) | Yes |
| Miglyol:Transcutol (95:5) | Yes |
| Miglyol:Transcutol (96:4) | No |
| Miglyol:Ethanol (91:11) | Yes |
| Miglyol:Labrasol (79:21) | No |
| Miglyol:Labrasol (70:30) | No |
| Miglyol:Labrafil (70:20) | No |
| Miglyol:Capmul PG8 (86:14) | No |
| Miglyol:Capmul PG8 (88:11) | Yes |

### Example 2

It was desired to achieve 50 mg of progesterone suspended in a medium that can also solubilize 2 mg estradiol in a total capsule fill mass of 200 mg. In order to achieve this formulation, the required solubility of estradiol needs to be ˜10 mg/g. A total fill weight of 200 mg was considered suitable for a size 5 oval soft gelatin capsule.

Additional solubility studies were performed to find solvent mixtures that might possibly be more suitable for soft gelatin encapsulation. Solubility studies were conducted with CAPMUL PG8 and CAPMUL MCM by mixing estradiol with various solvent systems and as before by analyzing for the amount of estradiol dissolved by HPLC after filtration. Results of these experiments are presented in Table 2. It can be seen from these results that mixtures containing MIGLYOL:CAPMUL PG8 at 50%; and also CAPMUL MCM alone or in combination with 20% Polysorbate 80 can achieve sufficient solubility to meet the target of 10 mg/g. CAPMUL PG8 mixed with MIGLYOL at the 15 and 30% level did not provide sufficient solubility.

TABLE 2

| Ingredient | Solubility (mg/g) |
|---|---|
| MIGLYOL:CAPMUL PG8 (85:15) | 4.40 |
| MIGLYOL:CAPMUL PG8 (70:30) | 8.60 |
| TRANSCUTOL:MIGLYOL 812:CAPMUL PG8 | >12 |
| (5:65:28) | |
| TRANSCUTOL:MIGLYOL 812:CAPMUL PG8 | >12 |
| (5:47:47) | |
| MIGLYOL:CAPMUL PG8 (50:50) | 14.0 |
| CAPMUL MCM | 19.8 |
| Polysorbate 80:CAPMUL MCM (20:80) | 15.0 |

Further solubility studies were performed using estradiol in various solvents and/or carriers, as shown in Table 2.1

TABLE 2.1

| | | Amount | | Soluble |
|---|---|---|---|---|
| Example | Ingredients | (mg) | % w/w | (Yes/No) |
| 2.1 | Miglyol | 1000 | 99.7 | No |
| | Estradiol | 3 | 0.3 | |
| 2.2 | Miglyol | 500 | 49.8 | Yes |
| | Transcutol | 500 | 49.8 | |
| | Estradiol | 4 | 0.4 | |
| 2.3 | Miglyol | 814.4 | 80.6 | Yes |
| | Transcutol | 189.0 | 18.7 | |
| | Estradiol | 7.3 | 0.72 | |
| 2.4 | Miglyol | 920.0 | 89.3 | Yes |
| | Transcutol | 102.8 | 10.0 | |
| | Estradiol | 7.5 | 0.7 | |
| 2.5 | Miglyol | 910.7 | 87.8 | Yes |
| | Transcutol | 118.3 | 11.4 | |
| | Estradiol | 8.8 | 0.8 | |
| 2.6 | Miglyol | 905.9 | 85.8 | Yes |
| | Capmul PG8 | 145.8 | 13.8 | |
| | Estradiol | 4.1 | 0.4 | |
| 2.7 | Miglyol | 915.2 | 88.4 | No |
| | Capmul PG8 | 112.9 | 10.9 | |
| | Estradiol | 7.4 | 0.7 | |

### Example 3

Additional studies were performed to assess the stability of estradiol (4-6 mg) in solvent mixtures, as reported in Table 3. MIGLYOL 812 with 4% TRANSCUTOL precipitated on Hot/Cold cycling after 96 hours, while estradiol solubilized in MIGLYOL:CAPMUL blends at 30 and 50% or in CAPMUL MCM alone, did not precipitate under the same conditions for a minimum of 14 days.

TABLE 3

| | Estradiol | Results Hot/Cold |
|---|---|---|
| Formulation | mg/g | Cycling |
| TRANSCUTOL:MIGLYOL | 4 | Crystallizes after |
| 812 (4:96) | | 96 hours |
| MIGLYOL 812:CAPMUL | 6 | Clear, after 14 days |
| PG8 (70:30) | | |
| MIGLYOL 812:CAPMUL | 6 | Clear, after 14 days |
| PG8 (50:50) | | |
| TRANSCUTOL:MIGLYOL | 6 | Clear, after 14 days |
| 812:CAPMUL PG8 (5:80:15) | | |
| CAPMUL MCM | 6 | Clear after 14 days |

12 mg estradiol solubilized in MIGLYOL:CAPMUL PG8 50:50, CAPMUL MCM, and in mixtures of TRANSCUTOL: MIGLYOL: CAPMUL PG8 are stable and do not precipitate for at least 12 days.

TABLE 4

| | Estradiol | Results Hot/Cold |
|---|---|---|
| Formulation | mg/g | Cycling |
| MIGLYOL 812:CAPMUL PG8 | 12 | Clear, after 12 days |
| (50:50) | | |
| TRANSCUTOL:MIGLYOL | 12 | Clear, after 12 days |
| 812:CAPMUL PG8 (5:65:28) | | |
| TRANSCUTOL:MIGLYOL | 12 | Clear, after 12 days |
| 812:CAPMUL PG8 (5:47:47) | | |
| CAPMUL MCM | 12 | Clear after 12 days |

### Example 4

In addition to determining physical stability of the estradiol solutions over time, it is necessary to determine if the fill material will be stable during the encapsulation process. One way to test these preparations is with the addition of water to the fill mass. As can be seen in Table 5, estradiol solutions at a concentration of 6 mg/g in Polyethylene Glycol 400 and CAPMUL MCM are able to absorb a minimum of 7% water without recrystallization, whereas the same concentration in MIGLYOL 812:CAPMUL PG8 (75:25) precipitates.

Estradiol solutions at a concentration of 12 mg/g in Polyethylene Glycol 400 and CAPMUL MCM are able to absorb a minimum of 7% water without recrystallization. All CAPMUL PG8 containing formulations turned hazy on the addition of water. However, it should be noted that estradiol recrystallization was not observed, and the addition of water to CAPMUL PG 8 alone (without any estradiol) also turns hazy on the addition of water.

TABLE 5

| | | Results after |
|---|---|---|
| | Estradiol | addition |
| Formulation | mg/g | of 7% water |
| MIGLYOL 812:CAPMUL | 6 | Precipitated |
| PG8 (75:25) | | |
| MIGLYOL 812:CAPMUL | 12 | Hazy |
| PG8 (50:50) | | |
| TRANSCUTOL:MIGLYOL | 12 | Hazy |
| 812:CAPMUL PG8 (5:65:28) | | |
| CAPMUL MCM | 12 | Clear |
| TRANSCUTOL:MIGLYOL | 12 | Hazy |
| 812:CAPMUL PG8 (5:47:47) | | |
| Polyethylene Glycol | 12 | clear |
| 400 | | |

In accordance with various embodiments, formulations in accordance with various embodiments have an exemplary shelf life of 3 months with storage at 25±2° C./60±5% RH in 75 cc HOPE white, opaque bottles with a 38/400 mm white child resistant cap.

Stability studies are conducted comprising various formulations in accordance with exemplary embodiments, including:

TABLE 5.1

| Item | | | |
|---|---|---|---|
| No. | Ingredient | % w/w | Mg/Capsule |
| 1 | Estradiol Hemihydrate USP | 0.64 | 2.07 |
| 2 | Medium chain triglycerides | 94.09 | 305.8 |
| 3 | Diethylene Glycol Monoethylether | 5.00 | 16.25 |
| 4 | Lecithin | 0.25 | 0.81 |
| 5 | Butylated Hydroxytoluene | 0.02 | 0.07 |

| | | |
|---|---|---|
| Total | 100.0 | 325.0 |

Packaging during testing comprises a 75 cc round HDPE bottle and 33 mm cap. A Brasken FPT 300F resin is associated with the cap. Testing criteria include visual appearance, assay of estradiol dissolution, content uniformity, and microbial limits testing.

Three test groups are created. Test group 1 comprises A test at 40° C./75% RH. Test group 2 comprises a test at 30° C./65% RH. Test group 3 comprises a test at 25° C./60% RH. Test group 1 is tested for visual Appearance, assay of estradiol, and dissolution at months 1, 2, 3, and 6. Test group 2 is tested for visual appearance, assay of estradiol, and dissolution at months 0, 1, 2, 3, 6, and 12. Test group 3 is tested for visual appearance, assay of estradiol, microbial limits, and dissolution at months 0, 1, 2, 3, 6, 12, and 24.

### Example 5

In an exemplary embodiment, a capsule is provided containing a fill material comprising:

TABLE 6

| | Mg/ |
|---|---|
| Ingredient | Capsule |
| Estradiol Hemihydrate | 2.00 |
| Triglyceride of caprylic/capric acid (e.g., MIGLYOL 812) | qs |
| Diethylene Glycol Monoethylether (TRANSCUTOL HP) | 65.00 |
| Liquid lecithin | 1.63 |
| Butylated Hydroxytoluene | 0.13 |
| Total Fill Weight | 325 |

### Example 6

In an exemplary embodiment, a capsule is provided containing a fill material comprising:

TABLE 7

| Ingredient | Mg/Capsule |
|---|---|
| Estradiol Hemihydrate | 2.00 |
| Monoglycerides/diglycerides of | qs |
| capric acid (e.g., CAPMUL MCM) | |
| Liquid lecithin | 1.63 |
| Polysorbate 80 | 97.5 |
| Total Fill Weight | 325 |

In an exemplary embodiment, a capsule is provided containing a fill material comprising:

TABLE 8

| | Mg/ | | Amount/ | |
|---|---|---|---|---|
| Ingredient | Capsule | % w/w | Batch | |
| Estradiol Hemihydrate | 2.03 | 0.62 | 20.2 | g |
| Monoglycerides/diglycerides of | 322.97 | 99.38 | 3.23 | kg |
| capric acid (e.g., CAPMUL MCM) | | | | |
| Total | | 100 | 3.25 | kg |

The above formulation is prepared as follows: estradiol is added to CAPMUL MCM and mixed until dissolved.

### Example 7

### Progesterone Solubility

In various embodiments, both estradiol and progesterone may be dissolved in a solvent. In various embodiments, the solubility of both estradiol and progesterone will be such that a therapeutically effective dose may be obtained in a reasonably sized mass, generally considered to be between 1 mg and 1200 mg, preferably suitable for encapsulation in a size 3 to 22 oval or oblong capsule. For example, in various embodiments, 50 mg to 100 mg of progesterone may be dissolved in a volume of solvent; i.e., the solubility would be 50 mg to 100 mg per capsule. MIGLYOL was attempted, and while it can be considered a good carrier for progesterone, it alone did not provide a desirable level of solubilization of estradiol (e.g., solubility of 12 mg/g may be desirable in various embodiments). Thus, MIGLYOL may be used in embodiments comprising a suspension of progesterone, though MIGLYOL, standing alone, is not desirable for use in embodiments having fully solubilized progesterone and/or estradiol.

As can be seen in Table 9, the solubility of progesterone in CAPMUL MCM is ˜73 mg/g. Therefore, by suspending 200 mg progesterone in 400 mg of solvent, part of the dose (˜14%) is already dissolved and the remaining is still a suspension. In some aspects and embodiments, it is desired to minimize the partial solubility of progesterone in the formulation in order to minimize the possibility of recrystallization.

Based on 73 mg/g solubility, the capsule size required to make a capsule of 50 mg solubilized progesterone would be 685 mg. Therefore, it was shown that it would be feasible to make a 50 mg progesterone and 2 mg estradiol solubilized formulation. MIGLYOL had the lowest solubility, but that solvent is unable to dissolve the estradiol, therefore under further experiments, it was decided to proceed with the second lowest or CAPMUL MCM. It has also been found that 2 mg of estradiol may also be dissolved in 685 mg of CAPMUL MCM.

TABLE 9

| | Progesterone Solubility |
|---|---|
| Ingredient | (mg/g) |
| CAPMUL MCM | 73.4 |
| CAPMUL PG8 | 95 |
| MIGLYOL 812 | 27.8 |
| CAPMUL MCM:GELUCIRE 44/14 (9:1) | 86.4 |
| CAPMUL MCM:GELUCIRE 44/14 (7:3) | 70.5 |
| CAPMUL MCM:GELUCIRE 44/14 (6:3) | 57.4 |

In addition, it has been found that the solubility of progesterone in a solvent of CAPMUL MCM in combination with GELUCIRE 44/14 in a 9:1 ratio increases the solubility to approximately 86 mg/g. Therefore, in various embodiments, progesterone and/or estradiol may be dissolved in a CAPMUL MCM and GELUCIRE 44/14 system, wherein the ratio of CAPMUL MCM to GELUCIRE 44/14 is 9:1.

TABLE 10

| | Progesterone Solubility |
|---|---|
| Ingredient | (mg/g) |
| CAPMUL MCM:GELUCIRE 44/14 (9:1) | 86.4 |
| CAPMUL MCM:GELUCIRE 44/14 (7:3) | 70.5 |
| CAPMUL MCM:GELUCIRE 44/14 (6:4) | 57.4 |

In an exemplary embodiment, a capsule is provided containing a fill material having fully solubilized progesterone and estradiol comprising:

TABLE 11

| | | | Qty/Capsule |
|---|---|---|---|
| Ingredient | Mass (mg) | % w/w | (mg) |
| Progesterone, USP, micronized | 50.00 | 7.14 | 50.00 |
| Estradiol Hemihydrate, USP | 2.03 | 0.29 | 2.03 |
| CAPMUL MCM, NF | | 82.57 | 577.97 |
| GELUCIRE 44/14, NF | | 10.0 | 70.00 |
| TOTAL | | 100.00 | 700.00 |

A capsule such as that shown in Table 11 may be manufactured in any suitable manner. For the purposes of this Example, mixing may be facilitated by an impeller, agitator, or other suitable means. Also for the purposes of this Example, heating and/or mixing may be performed under an inert or relatively inert gas atmosphere, such as nitrogen gas N2. Mixing and/or heating for the purposes of this Example may be performed in any suitable vessel, such as a stainless steel vessel.

For example, CAPMUL MCM may be heated to between 30° C. to 50° C., more preferably from 35° C. to 45° C., and more preferably to 40° C.+/−2° C. GELUCIRE 44/14 may be added to the CAPMUL: MCM and mixed until dissolved. The addition may occur all at once or may occur gradually over a period of time. Heat may continue to be applied during the mixing of the GELUCIRE 44/14 and the CAPMUL MCM.

Heat may be removed from the GELUCIRE 44/14 and CAPMUL MCM mixture. Estradiol Hemihydrate may be added to the mixture. The addition may occur all at once or may occur gradually over a period of time. Micronized progesterone may then be added to the GELUCIRE 44/14, CAPMUL MCM and Estradiol Hemihydrate mixture until dissolved. The addition may occur all at once or may occur gradually over a period of time.

### Example 8

In an exemplary embodiment, a capsule is provided containing a fill material having suspended progesterone comprising:

TABLE 12

| | mg/ | | |
|---|---|---|---|
| Ingredient | Capsule | % | Function |
| Micronized Progesterone | 200.00 | 30.77 | Active |
| Medium Chain Triglyceride | qs | qs | Carrier |
| (MIGLYOL 812 or equivalent) | | | |
| Lecithin Liquid | 1.63 | 0.25 | Lubricant/Emulsifier |
| Butylated Hydroxytoluene | 0.13 | 0.02 | Antioxidant |
| (also referred to as “BHT”) | | | |

The above formulation is prepared as follows: MIGLYOL is heated to about 45° C. GELUCIRE 44/14 is added and mixed until dissolved. BHT is added and mixed until dissolved. Progesterone is suspended and passed through a colloid mill. The resultant fill mass can be used for encapsulation.

In an exemplary embodiment, a capsule is provided containing a fill material having partially solubilized progesterone comprising:

TABLE 13

| | Qty/ | | | Amount/ |
|---|---|---|---|---|
| | Capsule | | | Batch |
| Ingredient | (mg) | % w/w | Function | (kg) |
| Micronized Progesterone, | 200.00 | 33.33 | Active | 2.0 |
| USP | | | | |
| Monoglycerides/diglyc- | 394.0 | 65.67 | Carrier | 3.94 |
| erides/triglycerides of | | | | |
| caprylic/capric acid | | | | |
| (CAPMUL MCM) | | | | |
| Lauroyl polyoxyl-32- | 6.0 | 1 | Lubricant/ | 0.06 |
| glycerides (GELUCIRE | | | Emulsifier | |
| 44/14 or equivalent) | | | | |
| Total | 600.00 mg | 100 | | 6.0 kg |

For suspensions of progesterone and partially solubilized progesterone, GELUCIRE 44/14 may be added at 1% to 2% w/w to increase viscosity. The above formulation is prepared as follows: CAPMUL MCM is heated to about 65° C. GELUCIRE 44/14 is added and mixed until dissolved. Heat is removed. Progesterone is added and the mixture is passed through a colloid mill. The resultant fill mass can be used for encapsulation.

### Example 9

In an exemplary embodiment, a capsule is provided containing a fill material having suspended progesterone comprising:

TABLE 14

| | | mg/ | |
|---|---|---|---|
| Ingredient | % | Capsule | Function |
| Micronized Progesterone | 30.77 | 200.00 | Active |
| Medium Chain Triglyceride | 65.93 | 428.55 | Carrier |
| (MIGLYOL 812 or equivalent) | | | |
| Lauroyl polyoxyl-32-glycerides | 3.00 | 19.50 | Suspending Agent |
| (GELUCIRE 44/14 or equivalent) | | | |
| Butylated Hydroxytoluene | 0.03 | 1.95 | Antioxidant |
| Total | 100 | 650 | |

In various embodiments, amounts of MIGLYOL may be present in a range from about 35-95% by weight; GELUCIRE 44/14 from about 0.5-30% by weight; and BHT from about 0.01-0.1% by weight.

### Example 10

For the purposes of this Example, a particle size analysis is conducted by using the Beckman Device. A sample API comprising micronized progesterone in accordance with various embodiments is provided for analysis.

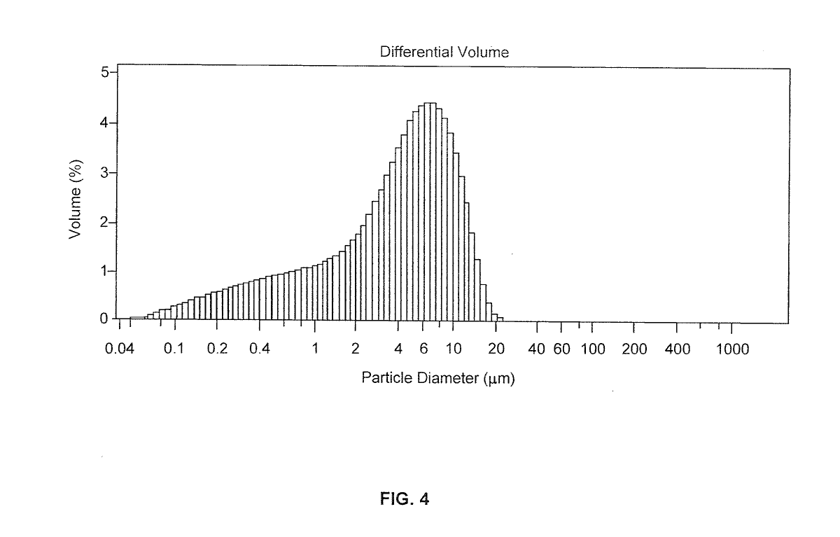

Approximately 0.01 g of a sample API in accordance with various embodiments was combined with Coulter 1B and 10 mL of deionized water. Sonication was performed for 15 seconds. The Beckman Device, equipped with a ULM, performed analysis for 90 seconds. The Beckman Device was configured to use the Fraunhofer optical model. The Beckman Device yielded that the sample has an X50 of 4.279 μM, an X75 of 7.442 μm, and an X25 of 1.590 μm. The Beckman Device also yielded that the mean particle size is 4.975 μm, the median particle size is 4.279 μm, the mode particle size is 6.453 μm, and the standard deviation is 3.956 μm. A graph of the particle distribution obtained is shown in FIG. 4.

### Example 11

A formulation sample having approximately 200 mg of micronized progesterone and 2 mg of estradiol was dispersed with oil. The Beckman Device, equipped with a MLM, performed analysis for 60 seconds. The Beckman Device was configured to use the Fraunhofer optical model. The Beckman Device yielded that the sample has an X50 of 11.0 μm, an X75 of 17.3 μm, and an X25 of 5.3 μm. The Beckman Device also yielded that the mean particle size is 11.8 μm, the median particle size is 11.04 μm, the mode particle size is 13.6 μm, and the standard deviation is 7.8 μm.

### Example 12

In order to increase the solubility of progesterone in the final solution, GELUCIRE 44/14 was added at about 10% w/w.

TABLE 15
Quantitative Formula: Batch Size 10,000 capsules

| | | Label | | Qty/ | Amount/ |
|---|---|---|---|---|---|
| Item | | Claim | | Capsule | Batch |
| No. | INGREDIENTS(S) | (mg) | % w/w | (mg) | (kg) |
| 1. | Progesterone, USP, | 50.00 | 7.14 | 50.00 | 0.50 |
| | micronized | | | | |
| 2. | Estradiol Hemihydrate, | 2.03 | 0.29 | 2.03 | 0.02 |
| | USP | | | | |
| 3. | CAPMUL MCM, NF | | 82.57 | 577.97 | 5.78 |
| 4. | GELUCIRE 44/14, NF | | 10.0 | 70.00 | 0.70 |
| | | Total: | 100.00 | 700.00 | 7.00 |

An example of the final formulation is provided in Table 15. The manufacturing process is as follows. CAPMUL MCM is heated to 40° C. GELUCIRE 44/14 is heated to 65° C. and added and mixed until dissolved. Heat is removed. Estradiol is added and mixed until dissolved. Micronized progesterone is then added and mixed until dissolved.

### Example 13

In an exemplary embodiment, a capsule is provided containing a fill material having fully solubilized estradiol and partially solubilized progesterone comprising:

TABLE 16

| | | Label | | Qty/ | Amount/ |
|---|---|---|---|---|---|
| Item | | Claim | | Capsule | Batch |
| No. | Ingredient(s) | (mg) | % w/w | (mg) | (g) |
| 1. | Progesterone, USP, | 50.00 | 25.000 | 50.00 | 500.00 |
| | micronized | | | | |
| 2. | Estradiol Hemihydrate | 0.25 | 0.129 | 0.26 | 2.58 |
| 3. | CAPMUL MCM, NF | | 73.371 | 146.74 | 1467.42 |
| 4. | GELUCIRE 44/14, NF | | 1.500 | 3.00 | 30.00 |
| | | Total: | 100.000 | 200.00 mg | 2000.00 |

The manufacturing process is as follows. CAPMUL MCM is heated to 65° C. GELUCIRE 44/14 is added and mixed until dissolved. Heat is removed. Estradiol is added and mixed until dissolved. Micronized progesterone is then added and dispersed. The mixture is then passed through a colloid mill. The resultant fill mass can be used for encapsulation.

### Example 14

In an exemplary embodiment, a capsule is provided containing a fill material having fully solubilized estradiol and partially solubilized progesterone comprising:

TABLE 17

| | | Label | | Qty/ | Amount/ |
|---|---|---|---|---|---|
| Item | | Claim | | Capsule | Batch |
| No. | Ingredient(s) | (mg) | % w/w | (mg) | (g) |
| 1. | Progesterone, USP, | 200.00 | 33.33 | 200.0 | 2000.0 |
| | micronized | | | | |
| 2. | Estradiol Hemihydrate | 2.00 | 0.35 | 2.07 | 20.7 |
| 3. | CAPMUL MCM, NF | | 65.32 | 391.93 | 3919.3 |
| 4. | GELUCIRE 44/14, NF | | 1.00 | 6.0 | 60.0 |
| | | Total: | 100.00 | 600.0 mg | 6000.0 |

The manufacturing process is as follows. CAPMUL MCM is heated to 65° C. GELUCIRE 44/14 is added and mixed until dissolved. Heat is removed. Estradiol is added and mixed until dissolved. Micronized progesterone is then added and dispersed. The mixture is then passed through a colloid mill. The resultant fill mass can be used for encapsulation. Alternatively, GELUCIRE 44/14 is heated to 65° C. and CAPMUL MCM is heated to 40° C.+/−5° C. to achieve mixing of the oil and the surfactant before heat is removed; estradiol is added while the mixture is cooling; progesterone is added when the mixture has dropped below about 40 C; the mixture is then passed through a colloid mill, e.g., three times.

### Example 15

### Study 352

### Progesterone and Estradiol Combination Study Under Fed Conditions

This following study protocol was used to establish bio-availability and bio-equivalence parameters for a combination product of the present disclosure comprising progesterone (200 mg) and estradiol (2.0 mg) as prepared via the process described in Example 14 and compared to 200 mg of PROMETRIUM (Catalent Pharmaceuticals, St. Petersburg, Fla. (and 2.0 mg of ESTRACE (estradiol vaginal cream, USP, 0.01%) (Bristol-Myers Squibb Co. Princeton, N.J.), administered to twenty-four (24) normal healthy, adult human post-menopausal female subjects under fed conditions.

The pharmaceutical formulation of the invention used in these PK studies had substantially the following formula:

| | | Qty/Capsule |
|---|---|---|
| Ingredient(s) | Amount (% w/w) | (mg) |
| Progesterone, | 7.14 | 50.00 |
| USP, | | |
| micronized | | |
| Estradiol | 0.30 | 2.07 |
| Hemihydrate, | | |
| USP | | |
| Micronized | | |
| CAPMUL | 83.27 | 582.93 |
| MCM, NF, | | |
| USP | | |
| GELUCIRE | 9.29 | 650 |
| 44/14, NF | | |
| Total | 100.00 | 700 |

The Study Design: An open-label, balanced, randomized, two-treatment, two-period, two-sequence, single-dose, two-way crossover study.

The subjects were housed in the clinical facility from at least 11.00 hours pre-dose to at least 48.00 hours post-dose in each period, with a washout period of at least 14 days between the successive dosing days.

Subjects were fasted for at least about 10.00 hours before being served a high-fat, high-calorie breakfast, followed by dosing, then followed by a 04.00 hour, post-dose additional period of fasting.

Standard meals were provided at about 04.00, 09.00, 13.00, 25.00, 29.00, 34.00 and 38.00 hours post-dose, respectively.

Water was restricted at least about 01 hour prior to dosing until about 01 hour post-dose (except for water given during dosing). At other times, drinking water was provided ad libitum.

Subjects were instructed to abstain from consuming caffeine and/or xanthine containing products (i.e. coffee, tea, chocolate, and caffeine-containing sodas, colas, etc.) for at least about 24.00 hours prior to dosing and throughout the study, grapefruit and\or its juice and poppy containing foods for at least about 48.00 hours prior to dosing and throughout the study.

Subjects remained seated upright for about the first 04.00 hours post-dose and only necessary movements were allowed during this period. Thereafter subjects were allowed to ambulate freely during the remaining part of the study. Subjects were not allowed to lie down (except as directed by the physician secondary to adverse events) during restriction period.

Subjects were instructed not to take any prescription medications within 14 days prior to study check in and throughout the study. Subjects were instructed not to take any over the counter medicinal products, herbal medications, etc. within 7 days prior to study check-in and throughout the study.

After overnight fasting of at least about 10.00 hours, a high-fat high-calorie breakfast was served about 30 minutes prior to administration of investigational product(s). All subjects were required to consume their entire breakfast within about 30 minutes of it being served, a single dose of either test product (T) of Progesterone 200 mg & Estradiol 2 mg tablets or the reference product (R) PROMETRIUM (Progesterone) soft gel Capsule 200 mg and ESTRACE® (Estradiol) Tablets 2 mg (according to the randomization schedule) were administered with about 240 mL of water under fed condition, at ambient temperature in each period in sitting posture. A thorough mouth check was done to assess the compliance to dosing.

All dosed study subjects were assessed for laboratory tests at the end of the study or as applicable.

In each period, twenty-three (23) blood samples were collected. The pre-dose (10 mL) blood samples at −01.00, −00.50, 00.00 hours and the post-dose blood samples (08 mL each) were collected at 00.25, 00.50, 00.67, 00.83, 01.00, 01.33, 01.67, 02.00, 02.50, 03.00, 04.00, 05.00, 06.00, 07.00, 08.00, 10.00, 12.00, 18.00, 24.00 and 48.00 hours in labeled K2EDTA—vacutainers via an indwelling cannula placed in one of the forearm veins of the subjects. Each intravenous indwelling cannula was kept in situ as long as possible by injecting about 0.5 mL of 10 IU/mL of heparin in normal saline solution to maintain the cannula for collection of the post-dose samples. In such cases, blood samples were collected after discarding the first 0.5 mL of heparin containing blood. Each cannula was removed after the 24.00 hour sample was drawn or earlier or if blocked.

At the end of the study, the samples were transferred to the bio-analytical facility in a box containing sufficient dry ice to maintain the integrity of the samples. These samples were stored at a temperature of −70° C.+20° C. in the bio-analytical facility until analysis.

Progesterone (Corrected and Uncorrected) and Estradiol (unconjugated) and estrone (total) in plasma samples is assayed using a validated LC-MS/MS method.

The pharmacokinetic parameters Cmax, AUC(0-t) & AUC(0-∞) were calculated on data obtained from 24 subjects for the test product and reference product. In general, bioavailability of progesterone and estradiol were similar but bioequivalence was not established.

Corrected pharmacokinetic profile summaries are presented in Table 18, below, for progesterone.

TABLE 18
Summary of Primary Pharmacokinetic Profile of Test Product (T)
versus Reference Product (R) for Progesterone (Corrected)

| | | | Arithmetic Mean ± Standard | |
|---|---|---|---|---|
| | Geometric Mean* | | Deviation | |
| | Test | Reference | Test | Reference |
| Pharmacokinetic | Product | Product | Product | Product |
| Parameter | (T) | (R) | (T) | (R) |
| Cₘₐₓ | 47.0 | 43.0 | 81.0 ± 82.8 | 117.7 ± 173.7 |
| AUC₀₋ₜ | 107.6 | 97.8 | 163.9 ± 136.5 | 191.1 ± 241.7 |
| AUC_{0-∞} | 110.7 | 110.0 | 173.5 ± 143.0 | 207.1 ± 250.3 |

*Estimate of Least Square Mean used to calculate Geometric Mean

### Study 351

### Progesterone and Estradiol Combination Study Under Fasting Conditions

Fasted studies using the above protocol and test and reference products were also conducted. However, rather than the high-fat meal prior to administration of the test and reference drug, each subject fasted for a period of at least twelve (12) hours prior to dose administration.

The pharmacokinetic parameters Cmax, AUC0-t & AUC0-∞ were calculated on data obtained from 23 subjects under fasting conditions for the test product and reference product. In general, bioavailability of progesterone and estradiol were similar but bioequivalence was not established.

Corrected pharmacokinetic profile summaries are presented in Table 19, below, for progesterone.

TABLE 19
Summary of Primary Pharmacokinetic Profile of Test Product (T)
versus Reference Product (R) for Progesterone (Corrected)

| | | | Arithmetic Mean ± Standard | |
|---|---|---|---|---|
| | Geometric Mean* | | Deviation | |
| | Test | Reference | Test | Reference |
| Pharmacokinetic | Product | Product | Product | Product |
| Parameter | (T) | (R) | (T) | (R) |
| Cₘₐₓ | 2.3 | 3.0 | 2.9 ± 2.3 | 3.9 ± 3.4 |
| AUC₀₋ₜ | 8.4 | 10.9 | 11.2 ± 8.7 | 14.5 ± 11.0 |
| AUC_{0-∞} | 12.9 | 17.2 | 15.1 ± 9.0 | 19.6 ± 10.2 |

*Estimate of Least Square Mean used to calculate Geometric Mean

The data indicate good (i.e., low) inter-patient and intra-patient variability relative to PROMETRIUM).

Bioequivalence studies comparing a 2-mg estradiol gelcap test product according to the present invention with 2 mg ESTRACE® (Estradiol) tablets (Bristol-Myers Squibb Co. Princeton, N.J.) are conducted under fed and fasting conditions as described above for the estradiol/progesterone formulations. Bioavailability improvements of the test product in reference to ESTRACE® are assessed.

### Example 16

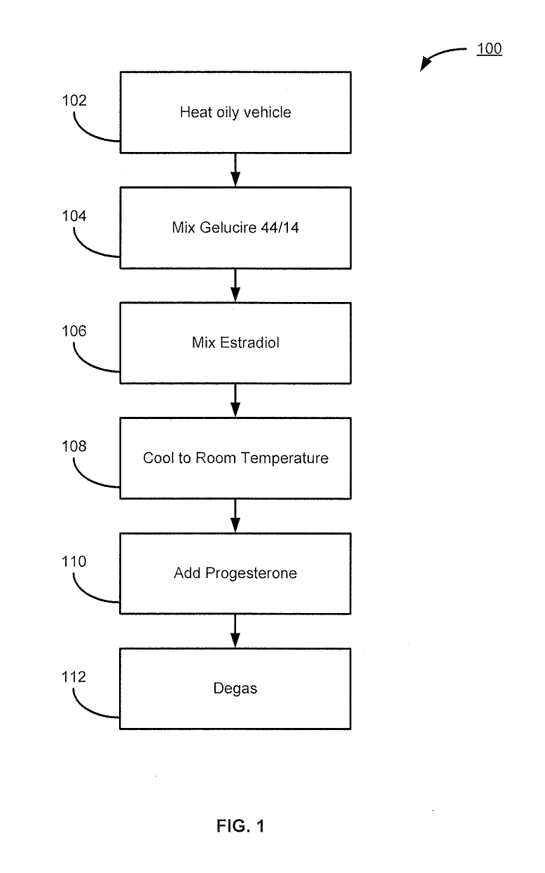

Method of manufacture in accordance with various embodiments are shown in FIGS. 1-3. With reference to FIG. 1, method of fill material 100 is shown. Step 102 comprises heating an oily vehicle carrier to 40° C.±5° C. Heating may be accomplished through any suitable means. The heating may be performed in any suitable vessel, such as a stainless steel vessel. The oily vehicle may be any oily vehicle described herein, for example, CAPMUL MCM.

Step 104 comprises mixing GELUCIRE 44/14 with the oily vehicle. Mixing may be facilitated by an impeller, agitator, or other suitable means. Step 102 may be performed under an inert or relatively inert gas atmosphere, such as nitrogen gas N2. Mixing may be performed in any suitable vessel, such as a stainless steel vessel.

Step 106 comprises mixing estradiol into the mixture of the oily vehicle and GELUCIRE 44/14. Mixing may occur in a steel tank or vat. Mixing may be facilitated by an impeller, agitator, or other suitable means. Step 106 may be performed under an inert or relatively inert gas atmosphere, such as nitrogen gas N2.

Step 108 comprises cooling to room temperature. Cooling may be allowed to occur without intervention or cooling may be aided by application of a cooling system.

Step 110 comprises mixing micronized progesterone into the mixture of oily vehicle, estradiol and GELUCIRE 44/14. Mixing may occur in a steel tank or vat. Mixing may be facilitated by an impeller, agitator, or other suitable means. Step 110 may be performed under an inert or relatively inert gas atmosphere, such as nitrogen gas N2. Step 112 comprises degassing. The resulting mixture from step 112 may comprise a fill material suitable for production into a softgel capsule.

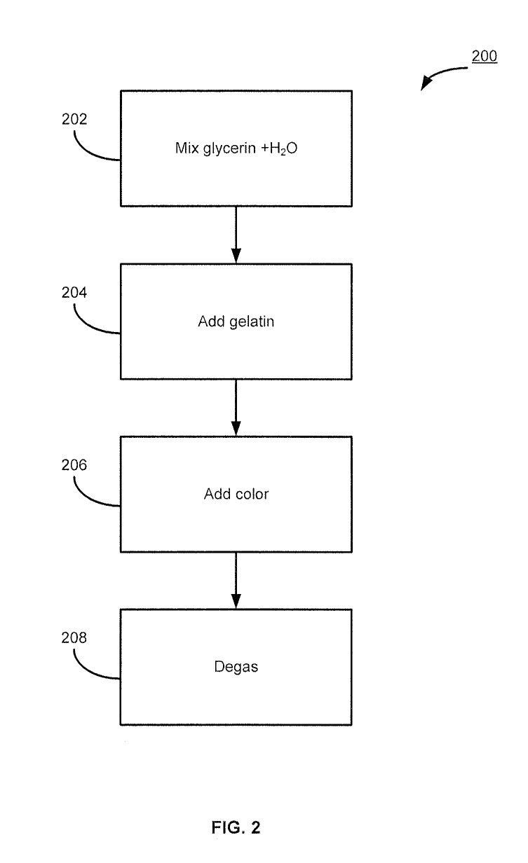

With reference to FIG. 2, softgel capsule, i.e. gel mass, production 200 is shown. Step 202 comprises mixing glyercin with water. The water used in step 202 may be purified by any suitable means, such as reverse osmosis, ozonation, filtration (e.g., through a carbon column) or the like. Mixing may be facilitated by an impeller, agitator, or other suitable means. Step 202 may be performed under an inert or relatively inert gas atmosphere, such as nitrogen gas N2. Heating may be performed until the temperature reaches 80° C.±5° C.

Step 204 comprises the addition of gelatin to the glycerin water mixture. Mixing may be facilitated by an impeller, agitator, or other suitable means. Step 204 may be performed under an inert or relatively inert gas atmosphere, such as nitrogen gas N2. A vacuum may be drawn in step 204 to de-aerate.

Step 206 comprises addition of a coloring agent such as a dye. A coloring agent may comprise products sold under the trademark OPATINT or other suitable agent. Step 206 may be performed under, an inert or relatively inert gas atmosphere, such as nitrogen gas N2.

Step 208 comprises degassing. The resulting mixture from step 208 may comprise a gel capsule material suitable for use as a gel capsule in production of a softgel capsule.

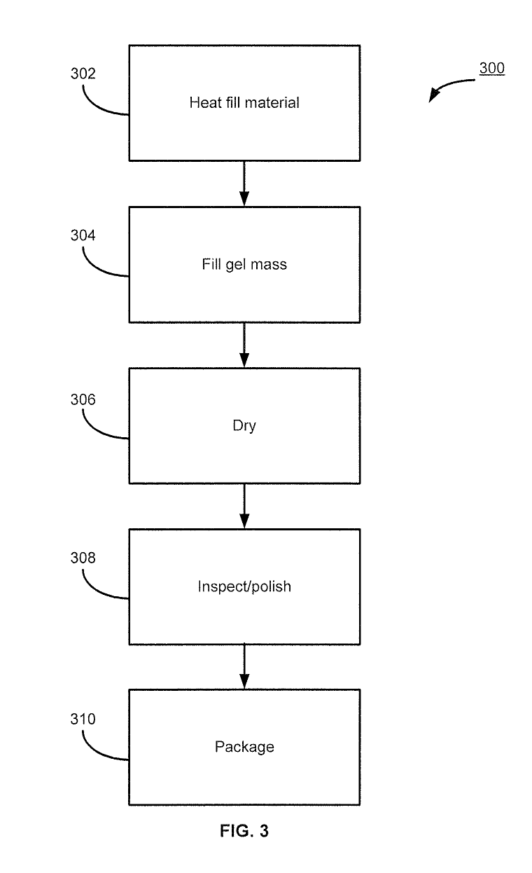

With reference to FIG. 3, softgel capsule assembly process 300 is shown. Step 302 comprises heating the fill material. The fill material may be heated to any suitable temperature. In various embodiments, the fill material is heated to 30° C.+/−3° C. Fill material maybe heated in a fill hopper. A fill hopper may comprise a device configured to hold a volume of the fill material and/or to dispense the fill material in controlled volumes.

Step 304 comprises filling a gel mass. A gel mass may be taken from the gel capsule material produced in step 208 of FIG. 2. Filling may be performed by injecting, placing, or otherwise disposing the fill material within a volume defined by the gel capsule material. The filling may occur in an encapsulator. The spreader boxes may be a temperature of 55° C.+/−10° C. The wedge temperature may be 38° C.+/−3° C. The drum cooling temperature may be 4° C.+/−2° C. The encapsulator may be lubricated using MIGLYOL 812 or other suitable lubricant. Step 304 thus produces one or more softgel capsules. Filling may comprise producing a ribbon of thickness 0.85 mm±0.05 mm using spreader box knobs. The fill material may be injected into the gel to produce a fill weight having target weight±5% (i.e., 650±33 mg and 325±16.3 mg).

Step 306 comprises drying the softgel capsules. Drying may be performed in a tumble dryer, tray dryer, or combinations thereof. For example, drying may be performed in a tumble drying basket for between about 10 minutes and about 120 minutes. Drying may continue in a drying room for about 24 hours to about 72 hours. Step 308 may comprise inspection and/or polishing. Polishing may be performed with isopropyl alcohol. Step 310 may comprise packaging. Packaging may be accomplished through any suitable means. Packaging may comprise packing softgel capsules into a blister pack, bottle, box, pouch, or other acceptable packaging.

### Example 17

### I. Solubility of Estradiol in Soy Bean Oil, Peanut Oil, and Safflower Oil

Data was obtained visually by making the mixtures described below, sonicating the mixtures, and then seeing if a clear solution resulted. If a clear solution was achieved, it was an indication of solubility at the level studied.

Procedures and Results:

Step 1.

0.3% of Estradiol suspension in each oil was prepared by adding 30 mg Estradiol to solvent and QS to 10 g. Samples were mixed on vortex for 2 hours, heated @ 50° C. for 30 minutes and then mixed for 1 hour more. All samples were still in suspension form.

Step 2.

Each sample was diluted to 0.24% (by adding 2.5 g more oil) and mixed for 2 hours and heated @ 50° C. for 30 min and mixed again for one hour. All the samples were still cloudy. Samples were kept at room temperature overnight to see if they precipitate or if un-dissolved API settles out. After 20 hours at room temperature, it was observed that all samples still had un-dissolved API.

Step 3.

Each sample was diluted to 0.2% (by adding 2.5 g more oil) and mixed 2 for hours and heated @ 50° C. for 30 min and mixed again for one hour. All the samples were still slightly cloudy, indicating that the estradiol was not completely dissolved.

TABLE 20

| | Estradiol Solubility | Estradiol Solubility |
|---|---|---|
| Ingredient | (mg/g) | (% w/w) |
| Peanut Oil | <2 | <0.2 |
| Safflower Oil | <2 | <0.2 |
| Soy Bean Oil | <2 | <0.2 |

The solubility of estradiol in all three oils was less than 2 mg/g (0.2% w/w). This level of solubility is significantly below the solubility that the present inventors have discovered can be achieved in other oils, e.g., medium chain fatty acid esters, such as the mono/diglycerides, propylene glycol esters, and polyethylene glycol esters discussed above.

In sum, if no heat is used to dissolve estradiol in safflower oil, it will not go into solution. Given that the estradiol did not dissolve at 50 C, oils such as safflower oil will not be useful in the methods of the invention using medium chain fatty acid esters as described hereinabove.

### II. Solubility in Safflower Oil

In a separate experiment, 50 g of safflower oil was heated to 85-88° C. and 60 mg estradiol was added, mixed until fully dissolved (1 hr), and allowed to cool to room temperature. The solubility achieved was 1.0 mg/ml. Addition of progesterone to a sample of the estradiol solution did not affect the solubility of estradiol.

Unsaturated fats are prone to oxidation, i.e., rancidity. Peroxides are intermediates formed during oxidation and the Peroxide Value is an indicator of extent of oxidation. The US Pharmacopeia specification for Peroxide Value of safflower oil is 10 max. Heating the oil, e.g., to 85° C., has been shown to increase the Peroxide Value. In contrast, medium chain fatty acid glycols, such as CAPMUL MCM and Myglyol 812, which comprise saturated C8-C10 fatty acid esters, have much lower Peroxide Values, e.g., on the order of 1 or less.

### Example 18

### Dissolution

Dissolution studies were performed using a formulation of this invention comparing the dissolution of progesterone to the dissolution of PROMETRIUM and comparing the dissolution of estradiol to the dissolution of ESTRACE. In one study, a formulation of the invention in capsules comprising 200 mg of progesterone and 2 mg estradiol was used. In a second study, a formulation of the invention in capsules comprising 50 mg of progesterone and 2 mg estradiol was used. The two formulations comprised:

The dissolution study was performed using a USP dissolution apparatus (reciprocating cylinder) (“USP Apparatus 3”). The apparatus was set to 30 dips per minute. 250 mL of a solution of 0.1N HCl with 3% sodium lauryl sulfate was used at 37 C.

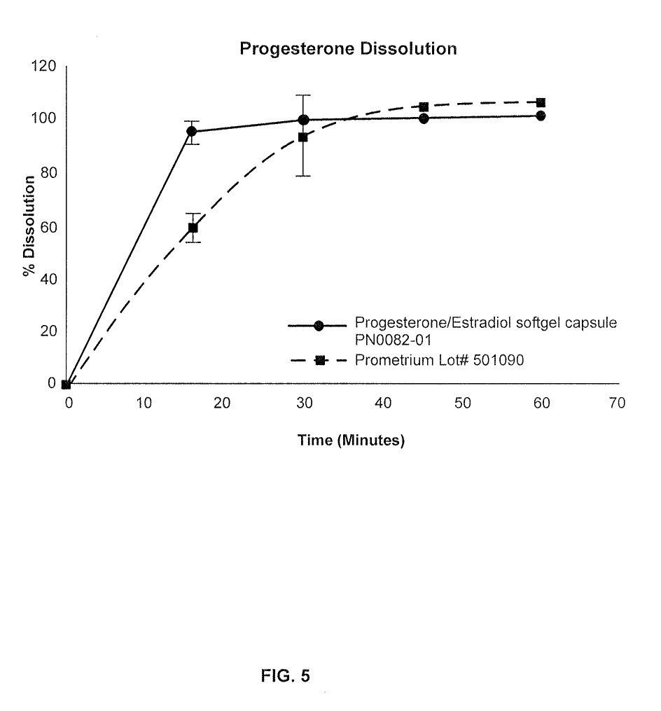

In both studies, progesterone was dissolved faster, and with smaller standard deviations, from the capsules of the invention than from PROMETRIUM. Dissolution of estradiol was comparable but marginally slower from the capsules of the invention than from ESTRACE. For illustrative purposes, a graph showing progesterone dissolution from the 200 mg progesterone capsule of the invention and from PROMETRIUM is attached as FIG. 5.

Both capsules of the invention were stable on storage in white HDPE bottles. Positive stability data were obtained with the 200 mg progesterone formulation over 6 months (>6 months data unavailable) and with the 50 mg progesterone formulation over 3 months (>3 months data unavailable).

It will be apparent to those skilled in the art that various modifications and variations can be made in the present disclosure without departing from the spirit or scope of the disclosure. Thus, it is intended that the present disclosure cover the modifications and variations of this disclosure provided they come within the scope of the appended claims and their equivalents.

Likewise, numerous characteristics and advantages have been set forth in the preceding description, including various alternatives together with details of the structure and function of the devices and/or methods. This disclosure is intended as illustrative only and as such is not intended to be exhaustive. It will be evident to those skilled in the art that various modifications may be made, especially in matters of structure, materials, elements, components, shape, size and arrangement of parts including combinations within the principles of the disclosure, to the full extent indicated by the broad general meaning of the terms in which the appended claims are expressed. To the extent that these various modifications do not depart from the spirit and scope of the appended claims, they are intended to be encompassed therein.

1. A composition comprising solubilized estradiol and a solubilizer comprising a medium chain oil, wherein the estradiol is at least 80% solubilized in the solubilizer, wherein the medium chain oil comprises substantially C6-C12 fatty acid esters, and wherein the composition is for oral administration.

2. The composition of claim 1, wherein the medium chain oil comprises a monoglyceride, a diglyceride, a triglyceride, or a mixture thereof.

3. The composition of claim 2, wherein the monoglyceride, diglyceride, or triglyceride comprises at least one of a caprylic acid ester and a capric acid ester.

4. The composition of claim 3, wherein the medium chain oil comprises a monoglyceride, a diglyceride, or a mixture thereof.

5. The composition of claim 1, further comprising 2-(2-ethoxy)-ethoxyethanol.

6. The composition of claim 1, wherein the solubilizer comprises a glycerol ester of saturated coconut or palm kernel oil.

7. The composition of claim 1, the composition having an estradiol dosage of about 0.25 mg.

8. The composition of claim 1, the composition having an estradiol dosage of about 0.5 mg.

9. The composition of claim 1, the composition having an estradiol dosage of about 1 mg.

10. The composition of claim 1, the composition having an estradiol dosage of about 2 mg.

11. The composition of claim 1, wherein the composition is in a capsule.

12. The composition of claim 11, wherein the capsule is a soft gelatin capsule or a soft vegetable-based capsule.

13. The composition of claim 11, wherein the capsule comprises:
a total fill weight between about 250 mg and about 400 mg, and
a weight ratio of estradiol to total fill weight from about 1 mg:250 mg to about 1 mg:60 mg.

14. The composition of claim 11, wherein the capsule comprises:
a total fill weight of about 325 mg, and
an estradiol dosage of about 2 mg.

15. The composition according to claim 1, wherein the composition provides increased bioavailability compared to ESTRACE (2 mg estradiol tablets, USP).

16. The composition according to claim 1, wherein the composition provides more consistent pharmacokinetics among subjects compared to ESTRACE.

17. The composition according to claim 1, wherein the composition provides improved content uniformity within the formulation compared to ESTRACE.

18. A method for treating menopausal-related symptoms, the method comprising administering to a subject in need of treatment the composition of claim 1.
